(19) United States
(12) Patent Application Publication
(10) Pub. No.: US 2021/0340200 A1
(43) Pub. Date: Nov. 4, 2021

(54) CD40L ANTAGONIST AND USES THEREOF

(71) Applicant: Viela Bio, Inc., Gaithersburg, MD (US)

(72) Inventors: Jorn DRAPPA, Gaithersburg, MD (US); Marius ALBULESCU, Gaithersburg, MD (US); Jing LI, Gaithersburg, MD (US); Ethan GRANT, Gaithersburg, MD (US); Katie STREICHER, Gaithersburg, MD (US); Gabor ILLEI, Gaithersburg, MD (US); Liangwei WANG, Gaithersburg, MD (US); William REES, Gaithersburg, MD (US)

(21) Appl. No.: 17/279,772

(22) PCT Filed: Sep. 25, 2019
(86) PCT No.: PCT/US2019/052997

§ 371 Date: Mar. 25, 2021

Related U.S. Application Data
(60) Provisional application No. 62/736,851, filed on Sep. 26, 2018.
(60) Provisional application No. 62/747,552, filed on Oct. 18, 2018.
(60) Provisional application No. 62/758,060, filed on Nov. 9, 2018.
(60) Provisional application No. 62/853,575, filed on May 28, 2019.

(51) Int. Cl. C07K 14/47; A61P 37/06; C12N 9/26; C12N 9/16; C12N 15/86

(52) U.S. Cl. CPC C07K 14/4703; A61P 37/06; A61K 38/00; C12N 9/16; C12N 15/86; C12N 9/2408

(57) ABSTRACT

A human CD40L-specific Tn3 molecule and therapeutic uses thereof.

### BACKGROUND

The CD40/CD40L pathway plays a critical role in driving humoral immune responses and has been implicated in the pathogenesis of several autoimmune diseases. CD40 is constitutively expressed on a variety of antigen presenting cells, including dendritic cells (DCs), macrophages, and B cells (1), and can also be expressed on non-hematopoietic cells.

Expression of the CD40 ligand, CD40L (also known as CD154), is highly regulated and is mostly found on activated CD4+ T cells (2). CD40/CD40L interactions between B cells and activated T cells are essential for mounting effective humoral responses to T-dependent antigens (3-5). The CD40/CD40L axis drives B cell expansion, differentiation and isotype switching in vitro (6-9). In vivo, CD40 signaling is required for germinal center (GC) formation, somatic hyper mutation and the generation of memory B cells and long-lived plasma cells (10-13). CD40 or CD40L defects in humans lead to X-linked hyperimmunoglobulin syndrome, a disease characterized by impaired isotype class switching, which manifests as high levels of serum IgM with low to no detectable IgG, IgA or IgE and increased susceptibility to infections (14-16).

Clinical trials with compounds directed against CD40L have demonstrated the potential benefits of targeting the CD40 pathway in autoimmune disease. In a Phase II trial, a humanized 5c8 anti-CD40L antibody, BG9588, significantly reduced proteinuria and anti-dsDNA antibody titers in patients with proliferative lupus nephritis (17). Additional studies revealed that anti-CD40L treatment reduced circulating CD38^{hi} Ig-secreting cells as well as peripheral GC B cells present in active SLE patients (18, 19). Anti-CD40L monoclonal antibody (mAb) treatments were also shown to induce a profound response in a subset of patients with immune thrombocytopenia (ITP) (20).

Although anti-CD40L mAb treatments have been shown to have potential in clinical trials, their programs have been halted due to adverse thromboembolic events. While not precisely defined, one potential explanation for these unanticipated safety issues is the expression of FcγRIIa (or CD32a) on human, but not mouse, platelets (21). CD40L is also highly expressed on activated platelets (22), where concurrent antibody-mediated binding to both CD40L and FcγRIIa on adjacent cells has the potential to result in platelet aggregation. Mouse models support a role for FcγRIIa in anti-CD40L induced thrombocytopenia. In mice transgenic for human FcγRIIa, anti-CD40L mAb caused shock and thrombocytopenia (23). This effect was not observed in either wild-type mice or in transgenic mice injected with an aglycosylated version of the antibody, unable to engage FcγR.

To target CD40L, but without the potential complications associated with a mAb, a CD40L-specific Tn3 scaffold protein (24, 25) was generated. Tn3 proteins are derived from the third fibronectin type III domain of human tenascin-C and can be engineered to confer target specific-binding properties (26, 27). Fusion of a bivalent CD40L-specific Tn3 protein to human serum albumin (HSA) resulted in a molecule, i.e., VIB4920, that was able to bind human CD40L and prevent its interaction with CD40 receptor. Consistent with this disruption in CD40L/CD40 interaction, VIB4920 was able to potently inhibit activation and differentiation of human B cells in vitro by blocking CD40 signaling events.

There is a need in the art for a new therapeutic that significantly impacts humoral immune responses and treats autoimmune and/or inflammatory conditions. There is also a need in the art to induce immune tolerance to replacement therapies in patients in need thereof.

It has now been discovered that VIB4920 reduces clinical symptoms and other markers of disease when administered to patients suffering from an autoimmune/inflammatory disease or disorder. In particular, administration of VIB4920 to rheumatoid arthritis (RA) subjects at particular doses results in statistically significant reductions, compared to placebo, in titers of rheumatoid factor (RF) autoantibodies, Vectra DA biomarker score, and disease activity measured by DAS28-CRP.

### BRIEF SUMMARY

The description provides for a method for suppressing a B cell- and T cell mediated immune response in a subject. The method includes steps of administering a dose of between 500 mg and 3000 mg VIB4920 to a subject in need thereof and suppressing the B cell- and T cell-mediated immune response.

The description also provides a method for treating an autoimmune disease or disorder. The method includes steps of administering a dose of between 500 mg and 3000 mg VIB4920 to a subject in need thereof and thereby treating the autoimmune disease or disorder.

The description further provides a method for reducing a measure of RA disease activity in a patient being treated for RA. The method includes steps of administering VIB4920 to the patient and reducing the measure of RA disease activity in the patient. The measure of RA disease activity reduced may include one or more of DAS28-CRP, clinical disease activity index (CDAI), tender joint count, swollen joint count, patient's global assessment or physician's global assessment. The VIB4920 may be administered at a dose of between approximately 500 mg and 3000 mg.

The description also provides a method for reducing RF autoantibodies in a patient in treatment for RA. This method includes steps of administering VIB4920 at a dose of between approximately 500 mg and 3000 mg to the patient and reducing RF autoantibodies in the patient.

The description additionally provides a method for reducing a biomarker score in a patient in treatment for RA. The method includes steps of administering approximately 500 mg to 3000 mg VIB4920 to the patient and reducing the biomarker score in the patient. In such a method, the biomarker score may be one or more of plasma cell (PC) gene signature, Vectra-DA score, or serum C reactive protein (CRP) level.

The description also provides a method for reducing PC gene signature scores in a patient in need thereof. The method includes steps of administering VIB4920 to a patient in need thereof and reducing the PC gene signature score in the patient. The patient in need thereof may be a patient being treated for systemic lupus erythematosus, rheumatoid arthritis, myositis, antiphospholipid syndrome, autoimmune hepatitis, Sjogren's disease, or other autoimmune or inflammatory conditions, as well as transplantation and graft vs host disease. The VIB4920 administered to the patient in need thereof may be at a dose of approximately 500 mg to 3000 mg

The description further provides a method for reducing autoantibodies in a patient in treatment for an autoimmune disorder or allo-antibodies in the case of transplant. The method comprises steps of administering VIB4920 to a patient in need thereof and reducing the autoantibodies, or allo-antibodies, in the patient. In such a method, the patient is in treatment for an autoimmune disease characterized by presence of autoantibodies or the patient is in treatment to prevent transplant rejection. The patient is administered VIB4920 at a dose of approximately 500 mg to 3000 mg.

The description also provides a method for reducing inflammation in a patient. The method includes steps of administering VIB4920 to a patient in need thereof and reducing inflammation in the patient. The patient may be a patient being treated for an inflammatory disease or disorder, or may be being prophylactically treated for anticipated inflammation in response to an organ or tissue transplant. The VIB4920 may be administered at a dose of approximately 500 mg to 3000 mg.

The description further provides a method of inducing immune tolerance to a replacement therapy in a patient. The method includes steps of administering VIB4920 to a patient in need of a replacement therapy and inducing immune tolerance to the replacement therapy in the patient. The VIB4920 may be administered at a dose of approximately 1000 mg to 3000 mg.

### BRIEF DESCRIPTION OF FIGURES

FIG. 1A-1G provides a biochemical characterization of human CD40L-specific Tn3 clones, including that of VIB4920, a bivalent 342 clone fused to HSA. FIG. 1A shows the ability of a set of human CD40L-specific Tn3 clones to inhibit CD40-CD40L interactions as measured by Proteon. The percent inhibition shown is over a range of Tn3 concentrations. The average of duplicate wells is shown. FIG. 1B shows the ability of anti-CD40L Tn3 proteins to inhibit CD40L-mediated signaling via NFkB. HEK293 cells expressing CD40R and an NFkB-luciferase-reporter, were stimulated with recombinant CD40L overnight in the presence of anti-CD40L Tn3 proteins. Percent inhibition of luciferase activity is shown. Data represent the mean of duplicate wells. FIG. 1C shows inhibition of CD86 upregulation by Tn3 constructs at various concentrations. Human PBMCs were stimulated with recombinant CD40L, following pre-incubation with a CD40L Tn3 protein, and expression of CD86 was assessed by flow cytometry. The mean of duplicate wells is shown. FIG. 1D Tn3 molecules indicated were tested for their ability to inhibit CD40-CD40L interactions in an ELISA assay. Data represent the mean of duplicate wells. FIG. 1E provides data from the screening of clone 342 for binding to a panel of related TNF family members, including Fas, TNFalpha, TNFbeta and OX40L. Clone 342 was found to selectively bind CD40L. FIG. 1F Proposed structure of VIB4920 based on crystallization of 342 Tn3 and published crystal structure of HSA (1). FIG. 1G Cartoon presentation of CD40/CD40L and 342/CD40L structures aligned through a common CD40L molecule. CD40L shown in green, Tn3 in magenta and CD40 receptor shown in cyan.

FIG. 2A-2D provide structural characterization of human CD40L-specific Tn3 clone 342. FIG. 2A is a cartoon presentation of the trimeric 342/CD40L structure. Extracellular domain of CD40L is shown in green; 342 is magenta. FIG. 2B shows the interface between 342 and CD40L. Fragments of CD40L and 342 are shown in green and magenta tubes, respectively. Amino acids involved in hydrogen bonds indicated with sticks. Hydrogen bonds are shown with black dash lines with associated distances (A). Bonds with distance up to 3.5 Å are shown. FIG. 2C and FIG. 2D illustrate electrostatic surface potential of interacting surfaces of (FIG. 2C) CD40L and (FIG. 2D) 342 CD40L-specific Tn3. Molecules are turned nearly 90 degrees to show the interface and are semitransparent to allow visualization of amino acids involved in hydrogen bonding. Red color designates negatively charged surface and blue color indicates positive charge.

FIG. 3A-3G show how VIB4920 inhibits CD40 signaling and activation of human B cells, but does not induce platelet aggregation in ex vivo studies. FIG. 3A shows percent inhibition, by VIB4920, of NFkB luciferase signal in engineered HEK29 cells stimulated with CD40L overnight. Data represent mean of duplicate wells. One of two independent studies is shown. FIG. 3B shows how VIB4920 and an anti-CD40L mAb were able to inhibit CD86 upregulation of stimulated human PBMCs stimulated. Human PBMCs were stimulated with recombinant human megaCD40L and the percentage of CD19+/CD86+ cells was measured by flow cytometry at 24 hours. Data represents the mean of duplicate wells. FIG. 3C Human B cells were stimulated with IL-21 and megaCD40L in the presence of control or anti-CD40L (mAb or VIB4920 Tn3). B cell expansion was quantified on day 3. Dotted line represents ATP levels in unstimulated cells. Data shown are mean and SD of triplicate wells and are representative of two independent experiments. FIG. 3D and FIG. 3E show the effect of anti-CD40L (mAb or VIB4920 Tn3) on human B cells if left unstimulated (nil) or if stimulated with IL-21, anti-IgM and megaCD40L. PC number was quantified on day 7. Specifically, FIG. 3D shows effect of indicated molecules at day 7, on percent of IgD-CD38hi PCs on day 7. FIG. 3E shows effect on PC number on day 7 with the indicated molecules at the indicated concentrations. Data shown are mean and SD of triplicate wells and are representative of two independent experiments. ****=p<0.0001 by two-tailed unpaired Students t-test. FIG. 3F and FIG. 3G show effect of anti-CD40L (mAb or VIB4920 Tn3) molecules on washed human platelets incubated with pre-formed immune complexes; platelet aggregation, or lack thereof, was measured for 12-14 minutes. FIG. 3F shows percent aggregation. Where indicated, platelets were pre-incubated with anti-CD32a antibody for 5 minutes prior to addition of immune complex. Adenosine diphosphate (ADP) was used as a positive control for aggregation. FIG. 3G provides percent aggregation following incubation of platelets with immune complexes with VIB4920 (Tn3) or anti-CD40L mAb (5c8) at the indicated concentrations. Data are representative of two independent experiments.

FIGS. 4A and 4B. Mouse surrogate CD40L-specific Tn3 shows potent neutralizing activity in vivo in response to immunization. In both FIG. 4A and FIG. 4B mice were immunized with sheep red blood cells (SRBC) on day 0 and control or anti-CD40L Tn3 (M31-MSA) were administered daily from days 9 to 13. FIG. 4A provides percent of germinal center B cells in the spleen and lymph node as quantitated by flow cytometry on day 14. Dots represent individual animals and data are representative of two independent studies. ****=p<0.0001 by two-tailed unpaired Students t-test. FIG. 4B provides production of anti-sheep red blood cell IgG as quantified from the serum on day 14. Data represent mean and SEM of four animals per group (n=1 study).

FIGS. 5A and 5B: Study design for Phase 1a clinical study to evaluate VIB4920 safety in healthy volunteers. FIG. 5A shows the study cohorts for the Phase 1a study. FIG. 5B provides the Phase 1a study dosing and immunization strategy.

FIGS. 6A and 6B: In a Phase 1a study of healthy volunteers, VIB4920 demonstrated a favorable PK profile. FIG. 6A shows circulating levels of VIB4920 as determined by ELISA at the indicated time points. Dotted line represents the lower limit of sensitivity for the assay. Error bars represent standard deviation of the mean, which was not calculated for groups with N=2 subjects. FIG. 6B shows levels of soluble CD40L, as assessed by ELISA, in all dose cohorts at the indicated time points. Dotted line represents lower limit of detection for the assay.

FIG. 7: VIB4920 inhibits anti-drug antibodies (ADAs) at high doses in the Phase 1a study of healthy volunteers. The presence of ADAs was determined by ELISA. Each subject within each cohort is depicted by an individual line. Subjects with high ADAs (>480 median titer) are indicated with a magenta line; subjects with low ADAs (<480 median titer) are indicated with a dark blue line; subjects with undetectable ADAs are noted with a light blue line.

FIG. 8A-8C: VIB4920 inhibits B cell proliferation and TDAR in healthy human volunteers in a dose-dependent manner. Healthy volunteers were immunized with KLH 14 days prior to treatment with placebo or VIB4920 and were re-challenged 15 days post-dosing. FIG. 8A provides the anti-KLH IgG titers in the healthy volunteers over multiple time points and at different VIB4920 doses. FIG. 8B provides the anti-KLH IgM titers in the healthy volunteers over multiple time points and at different doses of VIB4920. IgG and IgM titers were measured by ELISA. FIG. 8C is a dose response model for inhibition of anti-KLH IgG at day 43.

FIG. 9A-9C: VIB4920 inhibits B cell proliferation and plasma cell responses in the reduction of the TDAR in healthy human subjects. FIG. 9A provides the detected frequency of proliferating B cells (Ki67+CD19+) in circulation as quantified by flow cytometry at various time points in volunteers receiving either placebo or high dose VIB4920 in the TDAR test study. FIG. 9B provides the detected frequency of class-switched memory B cells (Ki67+CD19+IgD-CD27+) in circulation as quantified by flow cytometry at various time points in volunteers receiving either placebo or high dose VIB4920 in the TDAR test study. FIG. 9C provides the PC signature score in whole blood, as evaluated by Taqman PCR. Mean and standard error expression values for placebo and high dose VIB4920 groups shown. *=P<0.05, **=P<0.01 vs placebo, by Mann-Whitney U test.

FIG. 10: Phase 1b study design to evaluate VIB4920 in RA patients. Arrow indicate doses of VIB4920 or placebo.

FIG. 11: Cohort demographics and clinical characteristics of VIB4920 phase 1b clinical trial RA patients.

FIG. 12: VIB4920 demonstrates an acceptable safety profile in RA patients. Shown are the most common TEAEs occurring in at least 2 subjects in a phase 1b study of RA subjects.

FIG. 13A-13C: VIB4920 demonstrates linear PK and a dose-dependent reduction in ADAs in a Phase 1b study of RA patients. FIG. 13A provides concentrations of circulating VIB4920 as determined by ELISA at the indicated time points. The dotted line represents the lower limit of sensitivity for the assay. Mean and standard error of the mean are shown. FIG. 13B provides the percent of subjects with a positive ADA titer, determined by ELISA, for each dosage administered, at any time point in the study. FIG. 13C provides the ADA titer, determined by ELISA, over time in subjects with detectable ADA.

FIG. 14A-14F: VIB4920 reduces disease index scores and autoantibodies in RA patients. FIG. 14A shows change in DAS28-CRP from baseline (mean and standard error indicated) assessed at indicated time points for as specified dose of VIB4920 or placebo. FIG. 14B shows change in CDAI from baseline (mean and standard error indicated) assessed at indicated time points for as specified dose of VIB4920 or placebo. FIG. 14C shows change in Patient Global Assessment from baseline (mean and standard error indicated) assessed at indicated time points for as specified dose of VIB4920 or placebo. FIG. 14D shows change in Physician Global Assessment from baseline (mean and standard error indicated) assessed at indicated time points for as specified dose of VIB4920 or placebo. FIG. 14E shows change in Vectra DA score from baseline (mean and standard error indicated) assessed at indicated time points for as specified dose of VIB4920 or placebo. FIG. 14F shows measurement of percent reduction in RF antibody titers, measured by ELISA, at the indicated time points for each indicated dose of VIB4920 or placebo.

FIG. 15A-15B: VIB4920 dose-dependently reduces DAS28-CRP scores and RF autoantibodies in RA patients. FIG. 15A shows difference between placebo, in DAS28-CRP score on day 85, and the indicated doses of VIB4920. A linear dose-response is shown; it was identified as the best fitting model for evaluating the relationship between VIB4920 dose and disease activity reduction. FIG. 15B shows percent reduction in RF autoantibodies over placebo at the indicated doses of VIB4920 on day 85. An Emax model is shown; it was determined to be the best fit for evaluating VIB4920 dose relationship to RF titers.

FIG. 16: VIB4920 improves DAS28 categories of treated RA patients. DAS28 categories at day 85 are shown. Fifty percent and seventy five percent of RA patients treated in the 1000 mg and 1500 mg groups, respectively, had low disease activity or remission at day 85.

FIG. 17A-17C: Impact of VIB4920 on tender/swollen joint counts and CRP in phase 1b study of RA subjects. FIG. 17A shows the change in RA subjects' tender joint count from base line at the indicated doses and time points. FIG. 17B shows the change in RA subjects' swollen joint count from baseline at the indicated doses and time points. FIG. 17C shows the change in RA subjects' ratio to baseline in CRP levels at the indicated doses and time points. Mean and standard error for each are shown.

FIG. 18: Amino acid sequence of a VIB4920 molecule.

FIG. 19A-19B: Amino acid sequences of clone 342 CD40L-specific Tn3 molecules.

FIG. 20: Amino acid sequence of a bivalent clone 342 CD40L-specific Tn3 molecule.

FIGS. 21A and 21B: Amino acid sequences of clone 309 CD40L-specific Tn3 molecule.

FIG. 22A-22F: VIB4920 reduces RA patient disease index scores and autoantibodies both during the 12 week VIB4920 dosing time period and the twelve week observation period following administration of last VIB4920 dose. FIG. 22A shows change in DAS28-CRP from baseline (mean and standard error indicated) assessed at indicated time points for as specified dose of VIB4920 or placebo. FIG. 22B shows change in CDAI from baseline (mean and standard error indicated) assessed at indicated time points for as specified dose of VIB4920 or placebo. FIG. 22C shows change in Patient Global Assessment from baseline (mean and standard error indicated) assessed at indicated time points for as specified dose of VIB4920 or placebo. FIG. 22D shows change in Physician Global Assessment from baseline (mean and standard error indicated) assessed at indicated time points for as specified dose of VIB4920 or placebo. FIG. 22E shows change in Vectra DA score from baseline (mean and standard error indicated) assessed at indicated time points for as specified dose of VIB4920 or placebo. FIG. 22F shows measurement of percent reduction in RF antibody titers, measured by ELISA, at the indicated time points for each indicated dose of VIB4920 or placebo.

FIG. 23A-23C: VIB4920 impacts tender/swollen joint counts and CRP in RA patients. VIB4920's impact was detectable in the phase 1b clinical trial in RA patients during both the dosing phase and the 12 weeks post-dosing observation time period. FIG. 23A shows the change in RA subjects' tender joint count from base line at the indicated doses and time points. FIG. 23B shows the change in RA subjects' swollen joint count from baseline at the indicated doses and time points. FIG. 23C shows the change in RA subjects' ratio to baseline in CRP levels at the indicated doses and time points. Mean and standard error for each are shown.

### DETAILED DESCRIPTION

Described herein are VIB4920 and its usefulness in methods for suppressing a B cell-mediated immune response, in methods for treating autoimmune diseases or disorders, in methods of reducing inflammation, in methods for reducing autoantibodies in a patient, in methods of reducing a measure of RA disease activity in a patient, in methods of reducing RF autoantibodies in a patient, in methods of reducing plasma cell gene signature scores in a patient and in methods of inducing immune tolerance to a replacement therapy in a patient.

If VIB4920 is used to treat an autoimmune disease or disorder, the VIB4920 may be used to treat alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, Sjogren's syndrome, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation, sepsis, rheumatoid arthritis, peritonitis, Crohn's disease, reperfusion injury, septicemia, endotoxic shock, cystic fibrosis, endocarditis, psoriasis, arthritis (e.g., psoriatic arthritis), anaphylactic shock, organ ischemia, reperfusion injury, spinal cord injury and allograft rejection. autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, graft-versus-host disease, Hashimoto's thyroiditis, idiopathic pulmonary fibrosis, idiopathic thrombocytopenia purpura (ITP), IgA nephropathy, juvenile arthritis, lichen planus, lupus erythematosus, Meniere's disease, mixed connective tissue disease, IgG4 mediated disease multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, Takayasu arteritis, temporal arteristis/giant cell arteritis, ulcerative colitis, uveitis, ANCA-associated vasculitides, other vasculitides such as dermatitis herpetiformis vasculitis, vitiligo, rejection of solid organ transplant, graft versus host disease, panel reactive antibody desensitization in kidney transplant recipients, islet cell transplantation and allogeneic hematopoetic stem cell transplantation, focal segmental glomerulosclerosis (FSGS), glomerulonephritides.

VIB4920 may, more specifically, be used to treat RA, systemic lupus erythematosus (SLE), myositis, antiphospholipid syndrome, autoimmune hepatitis, focal segmental glomerulosclerosis (FSGS), lupus nephritis, inflammatory myopathies, idiopathic thrombocytopenia purpura (ITP), systemic sclerosis, vasculitis, cutaneous lupus, autoimmune hemolytic anemia, myasthenia gravis, IgG4 related disease, or Sjogren's syndrome. Furthermore, VIB4920 may be used to treat graft-versus-host disease and/or to reduce or prevent rejection of organ or tissue transplants.

The treatment of the autoimmune disease or disorder may be in the form of suppressing a B cell- or T cell-mediated immune response, which may be a reduction of class-switched antibodies, a reduction in circulating B cell subsets, a reduction in plasma activity or a reduction in plasma cells and plasma cell gene signature. The treatment of the autoimmune disease or disorder may be a reduction in markers of inflammation. The markers of inflammation may be one or more of autoantibody levels, plasma cell (PC) or PC gene signature (signature characterized by expression of genes IGHA1, IGJ, IGKC, IGKV4-1 and TNFRSF17), circulating B cell subsets and class-switched antibodies. The treatment of the autoimmune disease or disorder may be a reduction of clinical signs and symptoms, such as those measured by a patient or physician global assessment. Clinical signs and symptoms may include one or more of arthritis, pain, fatigue, fever, malaise, rash, weakness, or signs of organ dysfunction such as proteinuria or loss of kidney function.

If the method is one of reducing autoantibodies in a patient in treatment for an autoimmune disorder, the autoantibodies may be antinuclear antibodies, e.g., in a patient in treatment for SLE, Sjogren's syndrome, an inflammatory myopathy, or systemic sclerosis. The antinuclear antibodies may be one or more of Anti-SSA/Ro or anti-SSB-La autoantibodies (SLE or Sjogren's syndrome), anti-dsDNA antibodies (SLE), Anti-Smith antibodies (SLE), anti-topoisomerase antibodies (systemic sclerosis), or anti-histone antibodies (SLE). If the method is one of reducing autoantibodies in a patient in treatment for an autoimmune disorder, the autoantibodies may be liver kidney microsomal type 1 antibodies, e.g., in a patient in treatment for autoimmune hepatitis. If the method is one of reducing autoantibodies in a patient in treatment for an autoimmune disorder, the autoantibodies may be anti-nicotinic acetylcholine receptor or anti-muscle-specific kinase antibodies, e.g., in a patient in treatment for myasthenia gravis. If the method is one of reducing antibodies in a patient in treatment for transplantation, the antibodies may be alloantibodies.

The reducing of the autoantibodies in the patient in treatment for an autoimmune disorder may be a reduction in percent of the autoantibodies to a level that is at least 20% less than that prior to administration of VIB4920. It may be to a reduction in percent of the autoantibodies to a level at least 30%, at least 40%, at least 50%, at least 60%, at least 70%, or at least 80% relative to levels of the autoantibodies prior to VIB4920 treatment. The reduction in the autoantibodies may be achieved by within a month to three months of initiation of VIB4920 administration.

If the autoimmune disease or disorder is RA, the treatment of rheumatoid arthritis may be a reduction of one or more of RF autoantibodies, anti-citrullinated peptide antibodies, Vectra DA biomarker score (Vectra DA biomarker score being a composite score of expression levels of interleukin-6, tumor necrosis factor receptor type I, vascular cell adhesion molecule 1, epidermal growth factor, vascular endothelial growth factor A, YKL-40, matrix metalloproteinase 1, MMP-3, CRP, serum amyloid A, leptin, and resistin), plasma cell (PC) signature, serum reactive C protein (CRP), DAS28-CRP, or clinical disease activity index (CDAI), or may be a reduction in number of tender joints, intensity of joint tenderness, number of swollen joints, or intensity of joint swelling. If the autoimmune disease or disorder is RA, the treatment may be achievement of ACR20, ACR50, or ACR70.

The treatment of the autoimmune disease or disorder may be characterized by a reduction of at least 20% of clinical symptoms of the disease or disorder, or by a reduction in inflammation, or by a reduction in biomarkers of the disease or disorder, relative to their levels prior to the treatment with VIB4920. The reduction of any of these symptoms, or inflammation, or biomarkers, may be a reduction in the symptoms, or inflammation or biomarkers of at least 50% relative to their levels prior to the initiation of treatment with VIB4920. The reduction may be such that the autoimmune disease or disorder is characterized as being in remission.

Further, if the autoimmune disease or disorder is rheumatoid arthritis, then the treating of the autoimmune disease or disorder may reduce RF autoantibodies in the patient to levels that are approximately at least 20%, at least 30%, at least 40%, at least 45%, at least 50%, at least 60%, at least 75%, or at least 80% relative to levels of RF autoantibodies prior to VIB4920 treatment. If the autoimmune disease or disorder is rheumatoid arthritis, then the treating the autoimmune disease or disorder may be a reduction of DAS28-CRP, and the reduction of DAS28-CRP may be such that there is an adjusted mean difference of at least −1.2, or at least −1.5, or at least −2.0 or at least −2.2. Additionally, if the autoimmune disease or disorder is rheumatoid arthritis, then the treating the autoimmune disease or disorder may be a reduction of Vectra DA biomarker score, the reduction may be an adjusted mean difference of at least −10.3, or at least −10.5, or at least −10.8.

If VIB4920 is used in a method of reducing inflammation, the inflammation may be the result of an inflammatory disease or disorder, or may be due to or in anticipation of injury, such as due to an organ or tissue transplantation procedure. If VIB4920 is used in a method of reducing inflammation in an inflammatory disease or disorder, the inflammatory disease or disorder may be inflammatory myopathy, or lupus nephritis, cutaneous lupus, RA, SLE, ITP, myositis, Sjogren's syndrome, vasculitis, systemic sclerosis, autoimmune hemolytic anemia, myasthenia gravis or focal segmental glomerulosclerosis. If VIB4920 is used in method of reducing inflammation, the inflammation may be due to or in anticipation of injury, such as due to an organ or tissue transplantation procedure.

If the VIB4920 is used in a method of inducing immune tolerance to a replacement therapy in a patient, the VIB4920 may induce the immune tolerance by reducing the patient's production of neutralizing antibodies to the replacement therapy. If the patient is naïve to the replacement therapy, or has otherwise not yet produced neutralizing antibodies to the replacement therapy, then the inducing immune tolerance may prevent the patient from producing neutralizing antibodies to the replacement therapy in the first instance. However, if the patient produces neutralizing antibodies to the replacement therapy, then the VIB4920 may induce the immune tolerance by reducing levels of the neutralizing antibodies to the replacement therapy produced by the patient. The patient's levels of the neutralizing antibodies produced to the replacement therapy may be reduced by at least 50%, at least 55%, at least 60%, at least 65%, at least 70, at least 75%, at least 80%, at least 85%, at least 90%, at least 95%, or to a level that is undetectable. The percent reduction in the patient's production levels of the neutralizing antibodies to the replacement therapy may be a comparison of, or may be determined by comparing, a first level of the neutralizing antibodies produced in response to the replacement therapy prior to administration of a first VIB4920 dose to a second level of neutralizing antibodies produced in response to the replacement therapy following administration of a first or a second or a third or a fourth or a fifth VIB4920 dose. Alternatively, the percent reduction in the patient's production levels of the neutralizing antibody to the replacement therapy may be a comparison of, or may be determined by comparing, peak neutralizing antibody levels produced in response to the replacement therapy prior to administration of a first VIB4920 dose to peak neutralizing antibody levels produced in response to the replacement therapy following administration of a first or a second or a third or a fourth or a fifth VIB4920 dose.

The immune tolerance induction to the replacement therapy in the patient may, alternatively or additionally, be a reduction in a T cell response to the replacement therapy. If the patient is naïve to the replacement therapy, or has received the replacement therapy but does not yet have a T cell immune response to the replacement therapy, then the VIB4920 may reduce the patient's T cell response by preventing formation of an initial T cell response to the replacement therapy. However, if the patient has an existing T cell response to the replacement therapy, then the VIB4920 may induce immune tolerance by reducing the existing T cell response to the replacement therapy. The T cell response to the replacement therapy may be reduced by at least 50%, at least 55%, at least 60%, at least 65%, at least 70, at least 75%, at least 80%, at least 85%, at least 90%, at least 95%, or to a level that is undetectable. The percent reduction in the patient's T cell response to the replacement therapy may be a comparison of, or may be determined by comparing, a first level of T cell response to the replacement therapy prior to administration of a first VIB4920 dose to a second level of T cell response to the replacement therapy following administration of a first or a second or a third or a fourth or a fifth VIB4920 dose. Alternatively, the percent reduction in the patient's T cell response to the replacement therapy may be a comparison of, or may be determined by comparing, a peak T cell response level to the replacement therapy prior to administration of a first VIB4920 dose to a peak T cell response level to the replacement therapy following administration of a first or a second or a third or a fourth or a fifth VIB4920 dose. Reduction of a T cell response may be characterized by a reduction in proliferation and/or stimulation of CD4+ T cells stimulated by the replacement therapy. Reduction of a T cell response may also be characterized by a reduction in a CD4-dependent CD8+ T cell response to the replacement therapy.

The replacement therapy to which the immune tolerance is induced may be a peptide or a protein replacement therapy. If the replacement therapy is a peptide or a protein therapy, it may be a Factor VIII or Factor IX therapy and it may be administered to treat a patient suffering from hemophilia. If the replacement therapy is a peptide or a protein therapy, it may be an enzyme replacement therapy (ERT). If the replacement therapy is an ERT, the replacement therapy may be agalsidase alfa or agalsidase beta, it may replace alpha-Galactosidase A, and it may treat a patient suffering from Fabry disease. If the replacement therapy is an ERT, the replacement therapy may be Iaronidase, it may replace alpha-L-Iduronidase, and it may treat a patient suffering from mucopolysaccharidosis (MPS) type 1 (also known as Hurler, Hurler-Scheie or Scheie syndrome, depending on its severity). If the replacement therapy is an ERT, the replacement therapy may be alglucosidase, it may replace alpha-glucosidase, and it may treat a patient suffering from Pompe disease. If the replacement therapy is an ERT, the replacement therapy may be idursulfase, it may replace iduronate-2-sufatase, and it may treat a patient suffering from MPS type II. If the replacement therapy is an ERT, the replacement therapy may be imiglucerase or velaglucerase alfa or taliglucerase alfa, it may replace beta-glucocerebrosidase, and it may treat a patient suffering from Gaucher disease. If the replacement therapy is an ERT, the replacement therapy may be Naglazyme arylsulfatase B, it may replace N-acetylgalactosamine-4-sulfatase, and it may treat a patient suffering from MPS VI. If the peptide or protein replacement therapy is a peptide or protein, the immune tolerance induction may reduce production of neutralizing antibodies to the peptide or protein and/or may reduce a T cell response to the peptide or protein by the patient.

Further, the replacement therapy to which the immune tolerance is induced may be a viral vector that comprises a nucleic acid encoding a therapeutic peptide or protein. If the replacement therapy is a viral vector that comprises a nucleic acid encoding a therapeutic peptide or protein the viral vector may be adenovirus vector, an adeno-associated virus vector, a retroviral vector, a pox virus, an alphavirus, a herpes simplex viral vector or any other viral vector capable of delivering a nucleic acid encoding a therapeutic peptide or protein to the patient's cells. The viral vector may be modified, e.g., by pseudotyping and/or to delete its wildtype genes and/or to include the nucleic acids encoding the therapeutic peptide or protein.

The therapeutic peptide or protein encoded by the nucleic acid of the viral vector may be the therapeutic peptide or protein Factor VIII or Factor IX, or it may be an ERT such as agalsidase alfa, agalsidase beta, idursulfase, iaronidase, alglucosidase alpha, imiglucerase, velaglucerase alfa, taliglucerase alfa, or Naglazyme arylsulfatase B.

Furthermore, if the replacement therapy is a viral vector that comprises a nucleic acid encoding a therapeutic peptide or protein, then the VIB4920 may induce the immune tolerance by reducing an immune response to the viral vector, or by reducing an immune response to the therapeutic peptide or protein encoded by the viral vector, or both. The VIB4920 may induce the immune tolerance to the viral vector by reducing neutralizing antibodies and/or a T cell response to the viral vector, either the vector itself or cells infected by the viral vector. The VIB4920 may additionally, or alternatively, induce immune tolerance to the replacement therapy comprising the viral vector by reducing neutralizing antibodies or a T cell response to the therapeutic peptide or protein encoded by a nucleic acid of the viral vector.

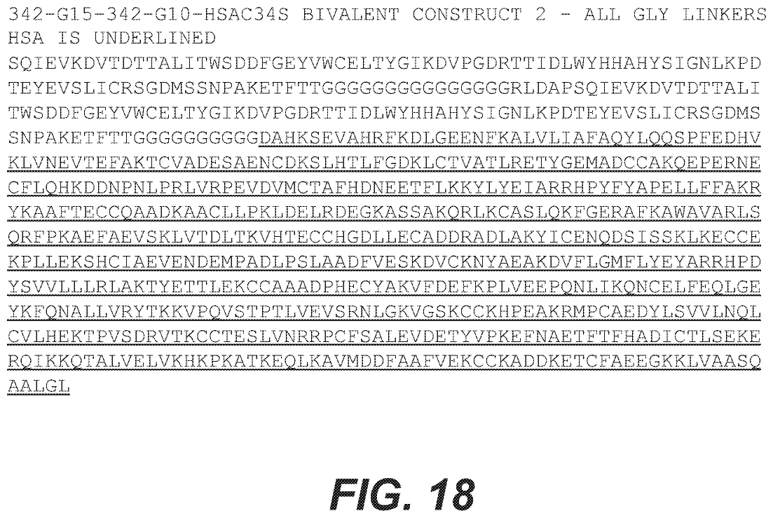

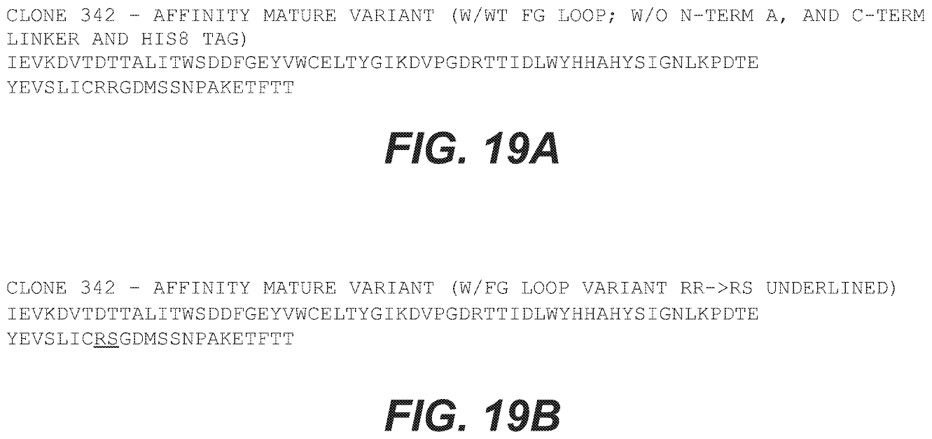

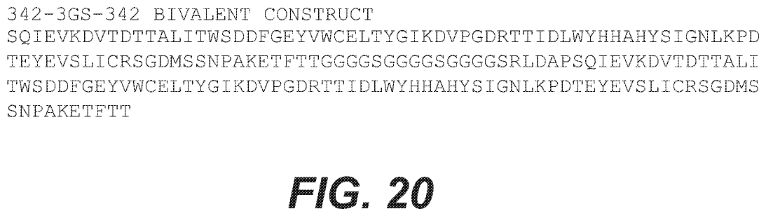

The VIB4920 for use in the various methods may comprise the amino acid sequence as shown in FIG. 18. The VIB4920 may have the amino acid sequence as shown in FIG. 18 or it may have one or more amino acid residues changes relative to the amino acid sequence as shown in FIG. 18. If the VIB4920 has amino acid sequence changes relative to those shown in FIG. 18, the changes may be to one of the linkers. VIB4920 comprises a Gly15 linker separating two CD40L-specific monomers and a Gly10 linker separating a CD40L-specific monomer from an HSA sequence. Both or one of these linkers may be altered, and may be replaced with an amino acid sequence of (GₘX)ₙ wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7. For example, one or both linkers may be altered to have an amino acid sequence that comprises one of GGGGSGGGGS, GGGGSGGGGSGGGGS, GGGGGGGGGG or GGGGGGGGGGGGGGG. If the VIB4920 has an amino acid sequence relative to the amino acid sequence as shown in FIG. 18, it may be due to a changes or changes in the HSA amino acid sequence fused to the two CD40L-specific monomers. The HSA fused to the two CD40L-specific monomers may be altered to relative to the HSA fused to the two CD40L-specific Tn3 monomers, except for at least one amino acid substitution, numbered relative to the position in full length mature HSA, at a position selected from the group consisting of 407, 415, 463, 500, 506, 508, 509, 511, 512, 515, 516, 521, 523, 524, 526, 535, 550, 557, 573, 574, and 580; wherein the at least one amino acid substitution does not comprise a lysine (K) to glutamic acid (E) at position 573. If the VIB4920 has amino acid sequence changes relative to those shown in FIG. 18, the changes may be to the amino acid sequence of one or both of the CD40L-specific Tn3 monomers, so long as it does not adversely effect in vivo efficacy of VIB4920, e.g., change in amino acid sequence such that one or both CD40L-specific Tn3 monomers have the amino acid sequence as shown in FIG. 19A.

The dose of VIB4920 administered in the methods may be a dose of between approximately 500 mg and approximately 3000 mg. The dose may be between approximately 750 mg and approximately 3000 mg, or between approximately 1000 mg and approximately 3000 mg, or between approximately 1500 mg and approximately 3000 mg, or between approximately 500 mg and approximately 2000 mg, or between approximately 750 mg and approximately 2000 mg, or between approximately 1000 mg and approximately 2000 mg, or between approximately 1000 mg and approximately 2500 mg, or between approximately 1000 mg and approximately 1500 mg. The dose may be 500 mg, 750 mg, 900 mg, 1000 mg, 1250 mg, 1500 mg, 1750 mg, 2000 mg, 2250 mg, 2500 mg, or 3000 mg.

The dose VIB4920 may be administered about every other week or may be administered twice per month. The dose VIB4920 may also be administered about every week or about once a month. The dose VIB4920 may be administered every 7 days, every 10 days, every 14 days, every 15 days, every 16 days, every 14-10 days, every 14-16 days, or every 30 days. The dose VIB4920 may be administered by intravenous or subcutaneous injection.

If the dose of VIB4920 administered is one of 1000 mg, 1500 mg, or between approximately 1000 mg and approximately 1500 mg, then the dose may be administered every other week or it may be administered twice per month. If the dose VIB4920 is 3000 mg, then the dose VIB4920 may be administered once per month. If the dose VIB4920 is 500 mg or 750 mg, then the dose VIB4920 may be administered once every other week, or, alternatively, be administered twice per month. Any of these doses may be administered intravenously.

The dose and dosing regimen of VIB4920 may be such that any therapeutic effect achieved from administration of VIB4920 to treat any autoimmune/inflammatory disease or disorder, e.g., reduction in autoantibodies, reduction in Vectra DA score, reduction in plasma cell signature, reduction in CRP, reduction in DAS28-CRP, reduction in swollen joint counts, reduction in tender joint counts, reduction in CDAI, improvement in patent's global assessment, improvement in physician's global assessment, achievement of ACR20, achievement of ACR50, or achievement of ACR70, may be considered to be “long-lasting.” A “long-lasting” effect of VIB4920 in the treatment of an autoimmune/inflammatory disease or disorder is one in which the therapeutic effect achieved by VIB4920 is maintained (although VIB4920 is no longer administered) over at least 4 weeks, at least 6 weeks, at least 8 weeks, at least 10 weeks, at least 12 weeks, at least 16 weeks, at least 20 weeks, or at least 24 weeks following administration of the last dose of a course of VIB4920. The course of VIB4920 may be administration of a dose of VIB4920 of between 500 mg and 3000 mg (e.g., 500 mg, 750 mg, 1000 mg, 1250 mgm 1500 mg, 1750 mg, 2000 mg, 2250 mg, 2500 mg, 2750 mg or 3000 mg) over a period of time of approximately between 8 and 24 weeks (e.g., 8 weeks, or 10 weeks, or 12 weeks, or 14 weeks, or 16 weeks, or 18 weeks, or 20 weeks, or 22 weeks, or 24 weeks, or 2 months or 4 months, or 6 months) at a dosing interval of once every 7 to 31 days (e.g., every 7 days, every 10 days, every 14 days, every 15 days, every 16 days, every 14-10 days, every 14-16 days, or every 30 days).

Those skilled in the art will recognize, or be able to ascertain using no more than routine experimentation, many equivalents to the specific embodiments described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

### EXAMPLES

### Example 1—Isolation and Optimization of CD40L-Specific Tn3 Proteins

Tn3 is a small protein scaffold, approximately 90 amino acids in length, that possesses immunoglobulin-like folds, including loops structurally analogous to antibody complementarity-determining regions, which can be randomized to select for specific binding properties (24).

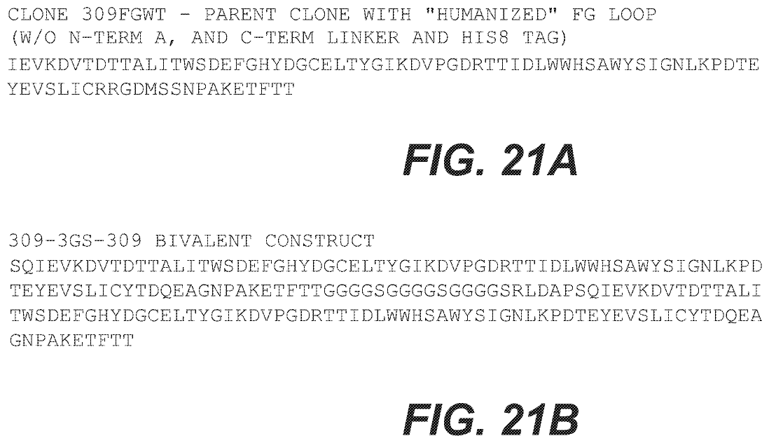

Human CD40L-specific Tn3 clones were isolated as described in detail in WO2013/055745 (see also 24, 27, 50). Briefly, selection of the human CD40L specific Tn3's included five rounds of panning, alternating between selection on recombinant human CD40L protein and a human CD40L-expressing CHO cell line. Murine CD40L-specific Tn3 proteins were selected using only recombinant mouse CD40L protein. Tn3 genes from selection outputs were pool-cloned into an expression vector, and individual His-tagged variants assessed for CD40L binding by capturing on Maxisorp plates coated with anti-His antibody (2 ug/ml in PBS). Biotinylated MegaCD40L was added (Enzo Biosciences, 0.5 μg/mL) and incubated for 1.5 hours. After washing once with PBS/Tween, the interaction between captured Tn3 variants and CD40L was monitored using SA-HRP (1:1000 dilution). After 20 minutes, plates were washed twice in PBS/Tween, developed with TMB substrate, and stopped with 2.5 M H3PO4. Absorbance was measured at 450 nm. Affinity maturation of CD40L-specific Tn3 proteins was performed by selection of improved candidates from phage displayed libraries in which the CDR-like loops were randomly mutated (24). This strategy led to the generation of clone 342 (FIG. 19), an improved variant of the human CD40L-specific 309 (FIG. 21A), and to clone M31, an improved variant of the murine CD40L-specific M13. Additional human CD40L-Tn3 clones, e.g., clones 304, 311, 320, 310, 321, and 322, were also prepared. See WO 2013/055745, hereby incorporated by reference.

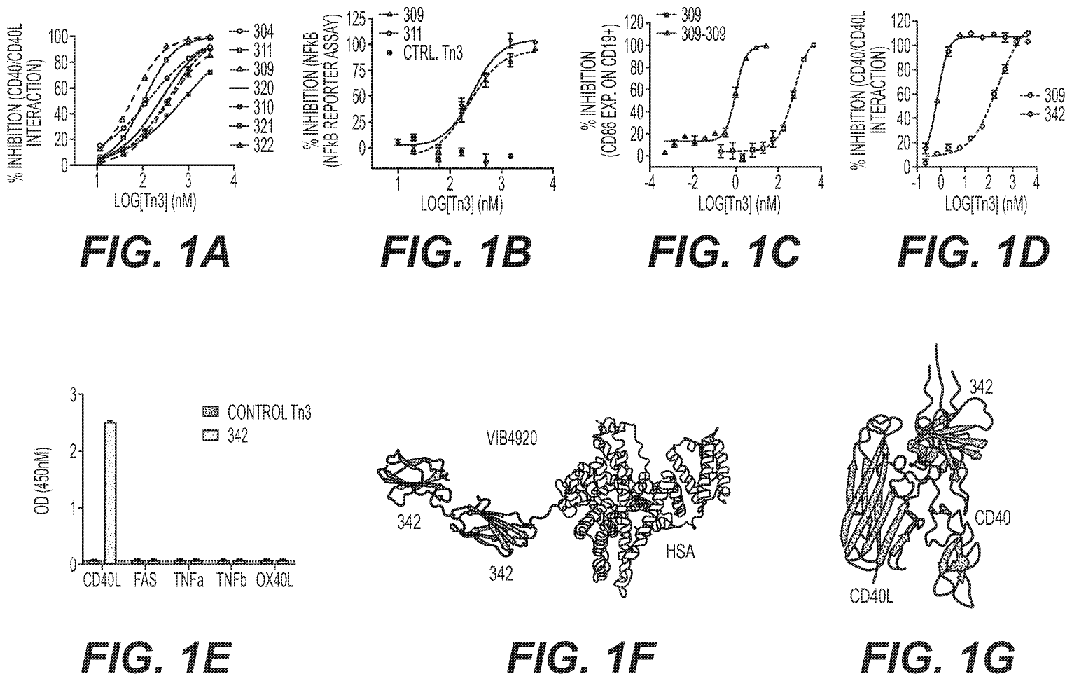

This set of human CD40L-specific Tn3 clones was characterized for its ability to biochemically inhibit binding of CD40L to its receptor (CD40). All seven of the set inhibited binding of CD40L to CD40, with IC50 values below 1 μM (FIG. 1A). The two most potent inhibitors in the biochemical CD40L-CD40 inhibition assay were further evaluated for inhibition of CD40L-mediated signaling in a cell-based reporter assay. HEK-293 cells expressing human CD40 and an NF-kB-luciferase reporter gene were stimulated with recombinant human CD40L protein. Human CD40L-specific Tn3 proteins 309 and 311 dose-dependently inhibited CD40L-induced NF-kB reporter gene expression at micromolar concentrations (FIG. 1B), highlighting the ability of these proteins to functionally inhibit CD40/CD40L signaling.

Simultaneous binding to multiple targets, as occurs in the case of bivalent antibodies, can result in markedly increased avidity. To explore the impact of bivalency on the potency of CD40L-specific Tn3 proteins, two copies of identical Tn3 modules (309-309; e.g., FIG. 21B) were linked via a flexible Gly4Ser containing spacer to form a tandem bivalent fusion protein. Human primary B cells upregulate the activation marker CD86 in response to stimulation through CD40. Pre-incubation of human peripheral blood mononuclear cells (PBMCs) with the monovalent CD40L-specific Tn3 309 inhibited upregulation of CD86 on human CD19+ B cells in a dose dependent manner (FIG. 1C). Strikingly, compared to the monovalent Tn3, there was a nearly 1000-fold improvement in potency in this primary cell assay using the bivalent construct (FIG. 1C). In addition, affinity maturation (through random mutagenesis in the variable CDR-like loop regions) of clone 309, resulting in clone 342, significantly improved binding affinity for CD40L (309: 190 nM; 342: 1.4 nM) and demonstrated an approximately 300-fold improved potency in inhibition of CD40-CD40L interactions (FIG. 1D). The affinity matured clone 342 was also screened for binding to a panel of related TNF family members, including Fas, TNFα, TNFβ and OX40L, and was found to selectively bind CD40L. See FIG. 1E.

Finally, as with other alternative scaffold technologies and due to their small size, naked Tn3 molecules would be expected to exhibit very rapid clearance from circulation when administered systemically. To improve the pharmacokinetic properties of the proteins, CD40-specific Tn3 proteins were fused to serum albumin (28, 29). The bivalent mouse surrogate CD40L-specific Tn3 protein, M13-M13, had a half-life, in mice when delivered systemically, of <30 minutes. Fusion of mouse serum albumin (MSA) to the M13-M13 Tn3 protein resulted in a 65-fold increase in serum half-life and 345-fold decrease in clearance (Table 1).

TABLE 1
Fusion with serum albumin greatly
improves half-life of M13-M13 Tn3 molecule

| Molecule | M13-M13 | M13-M13-MSA |
|---|---|---|
| Half life (days) | 0.02 | 1.3 |
| Cmax (μg/ml) | 10.7 | 135.8 |
| AUC (μg/day/ml) | 0.4 | 142.1 |
| CL (ml/day/kg) | 24305.2 | 70.4 |
| Vss (ml/kg) | 346.5 | 126.8 |

5-7 week old CD-1 mice received a single injection of bivalent CD40L-specific Tn3 molecule with or without MSA (n=12/group; 10 mg/kg, i.v.). Blood was sampled from n=3 mice/group at various time points between 15 minutes and 72 hours and circulating levels of Tn3 proteins were determined by ELISA.

Based on these observations, a bivalent human CD40L-specific Tn3 molecule, VIB4920, is comprised of tandem 342 CD40L-specific Tn3 proteins, for optimal potency, fused to human serum albumin (HSA), for improved half-life (FIG. 1F; FIG. 18).

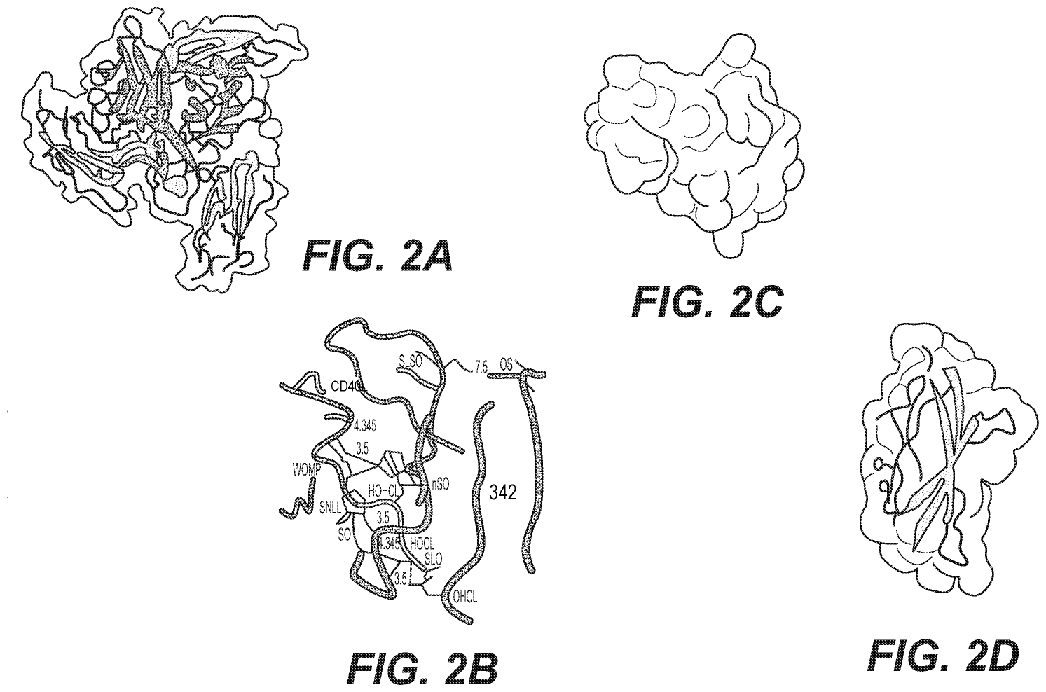

To better understand the molecular nature of the interaction between CD40L and VIB4920, crystallography studies were performed. CD40L-specific Tn3 (342) and soluble CD40L proteins were expressed, purified, co-crystallized, and the structure determined at 2.8 Å resolution. The molecular structure of trimeric soluble CD40L complexed with Tn3 is shown in FIG. 2A. The interface with CD40L is composed of amino acids mostly from the second modified loop of the Tn3, including eight of the ten hydrogen bonds formed between the molecules (within a distance of 3.5 Å, FIG. 2B). Initial characterization of the molecule indicated that the 342 Tn3 was able to block the interaction between CD40L and CD40. To visualize the details of that action the structure of the CD40/CD40L complex was superimposed with that of 342/CD40L (FIG. 1G). Superimposition shows that 342 and CD40 share a common binding site on CD40L. Hence, VIB4920 competes with CD40 and prevents its association with CD40L.

### Example 2—VIB4920 Blocks Activation and Differentiation of Human B Cells

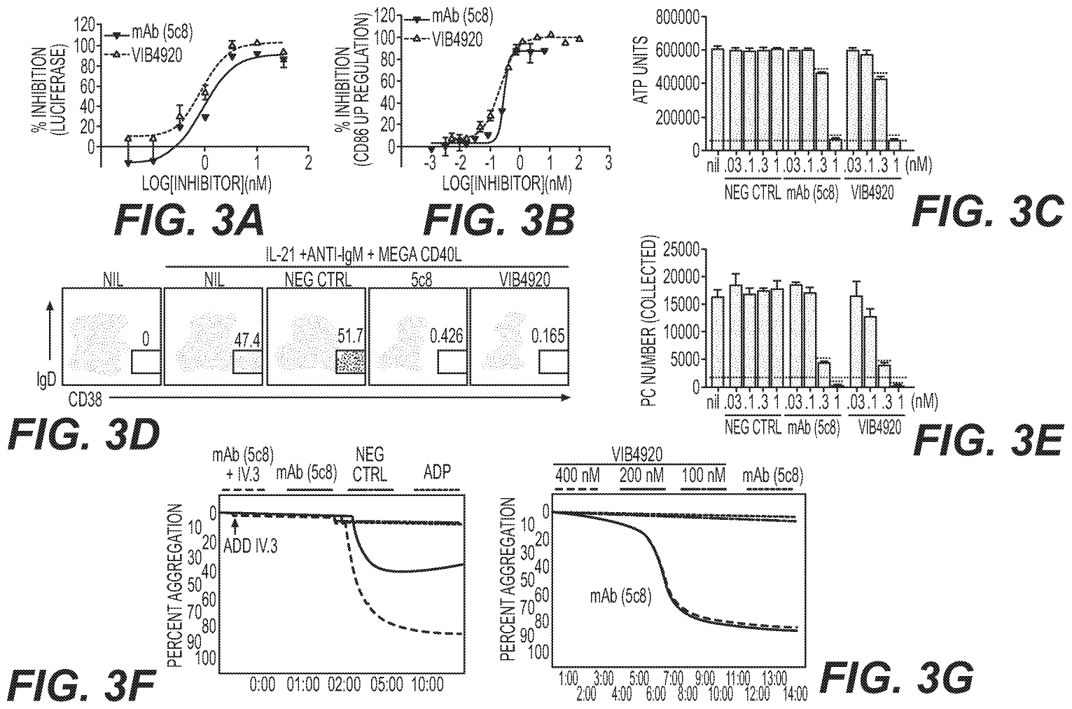

CD40 signaling has been extensively characterized and involves the activation of a variety of different pathways and transcription factors, including NF-kB (30), which can promote B cell activation, proliferation and differentiation (31). Thus, the ability of VIB4920 to inhibit CD40L-mediated activation of NF-kB was investigated using a cell line that expresses human CD40 and an NF-kB luciferase reporter gene. Stimulating this cell line with recombinant human CD40L or with CD40L-expressing cells, induces NF-kB activation. VIB4920 was able to potently block CD40 signaling using this cell line as evidenced by dose-dependent inhibition of NF-kB activation (IC50: 0.899 nM; FIG. 3A).

Resting B cells constitutively express low levels of the co-stimulatory molecule CD86, which is rapidly upregulated following activation, including activation through CD40 (32). Primary human PBMCs were stimulated with recombinant human CD40L and expression of CD86 was evaluated on B cells after 16 hours by flow cytometry. VIB4920 fully prevented CD40L-mediated upregulation of CD86 by primary human B cells (FIG. 3B).

### Example 3—VIB4920 does not Induce Platelet Aggregation In Vitro

Anti-CD40L-directed mAbs have failed in clinical trials due to safety concerns, largely due to thromboembolic complications related to cross-linking CD40L on the cell surface of platelets. To confirm that VIB4920, which lacks an Fc domain, does not induce platelet aggregation, we evaluated its impact on washed human platelets in vitro. As previously described, when pre-complexed with sCD40L, anti-CD40L mAb (human IgG1) showed a marked ability to induce platelet aggregation (FIG. 3F). The response was rapid, with mAb-sCD40L immune complexes inducing 80% of the platelets to aggregate within 8 minutes. Importantly, pre-incubation of platelets with an antibody which blocks FcγRIIa (mAb IV.3) prevented mAb-immune complex mediated aggregation, consistent with an essential role for Fc receptors in this response (FIG. 3F). By contrast, at several concentrations tested, VIB4920 showed no propensity to induce platelet aggregation in this assay (FIG. 3G). These data suggest that the absence of an Fc region in Tn3 constructs could reduce the risk of platelet aggregation and thromboembolic events that have been observed with therapeutic anti-CD40L antibodies in the clinic. In confirmation of this result, data (not shown) from a chronic (seven-month) study in nonhuman primates dosed with up to 300 mg/kg VIB4920 identified no adverse findings in platelet function, e.g., identified no adverse findings in D-dimer (to monitor for blood clots), PFA100 (to assess platelet function), or TAT complex (thrombin anti-thrombin complex) tests.

### Example 4—CD40L-Specific Tn3 Proteins Modulate Immune Responses In Vivo

The central role of CD40L in promoting T-dependent immune responses has been well characterized (9, 35). Therefore, a T-dependent immunization model was used to evaluate the ability of the Tn3-MSA fusion protein to block humoral immune responses in vivo. Due to insufficient sequence homology between human and murine CD40L, a CD40L-specific mouse surrogate Tn3, M31, was used for these studies.

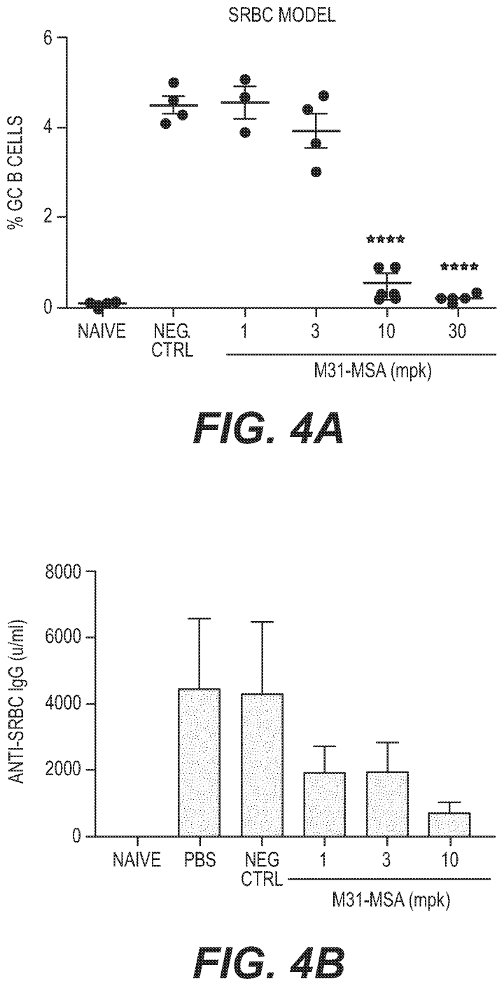

To test whether the Tn3-MSA fusion protein was able to block immune responses in vivo, mice were inoculated with sheep red blood cells (SRBCs) and then treated daily, on days 9-13 post-inoculation, with anti-CD40L Tn3 protein. The immune response in treated animals was assessed on day 14 by quantitating splenic and lymph node germinal center B cells by flow cytometry. As expected, immunization with SRBCs in control-treated mice led to a profound expansion of germinal center frequency (FIG. 4A). A dose-dependent reduction of germinal center B-cell frequency was observed in mice treated with the CD40L-specific Tn3-MSA fusion protein (FIG. 4A). At a dose of 30 mg/kg, the CD40L-specific Tn3-MSA fusion protein induced complete suppression of germinal center formation, as assessed by the near absence of germinal center B-cells in the spleen and lymph node, equivalent to control non-immunized mice. Other sub-populations of cells were not perturbed by drug administration, including specific T-cell populations, assuring that the effects observed were not secondary to T cell depletion (data not shown). In addition, anti-SRBC IgG levels mirrored that of the germinal B-cell response, with profound reductions in SRBC-specific Ig titers at higher doses of anti-CD40L Tn3 (FIG. 4B).

### Example 5—VIB4920 is Well Tolerated in Healthy Volunteers

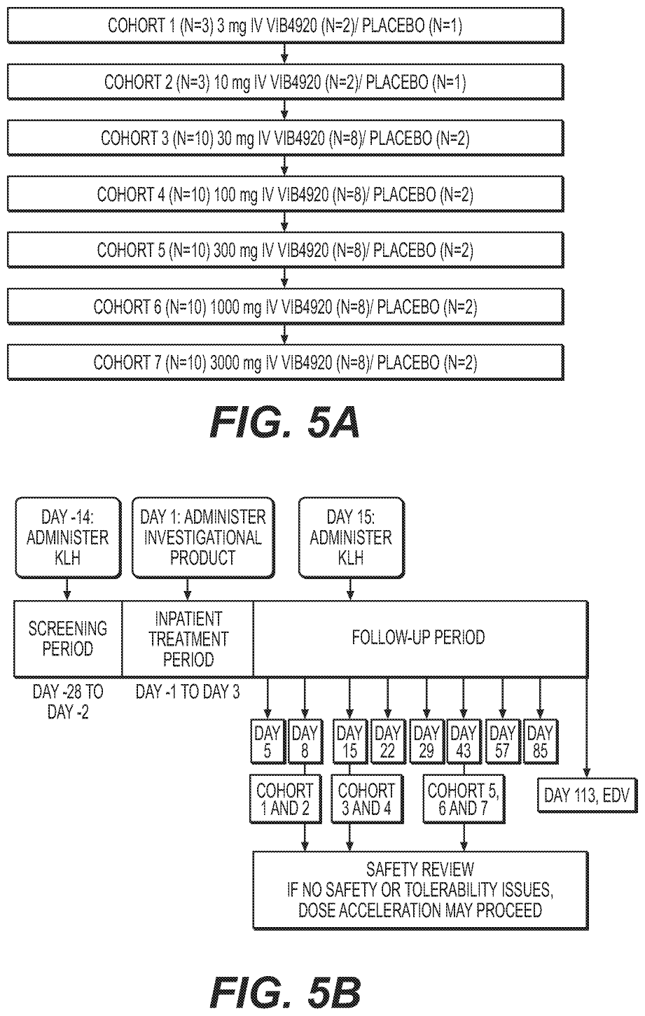

The safety properties of VIB4920 were evaluated in humans in a Phase 1a (Ph1a) study conducted in healthy adults aged 18-49 years. Subjects were enrolled into seven single dose-escalating cohorts with VIB4920 doses of up to 3000 mg, and randomized to VIB4920 or placebo (FIGS. 5A and 5B). The primary endpoint, safety and tolerability, was measured by the incidence of treatment-emergent adverse events (TEAEs) and treatment-emergent serious adverse events (TESAEs). In all dose cohorts, TEAEs were generally of minor clinical significance, with the most frequent events including nasopharyngitis (common cold) and headache (Table 2).

TABLE 2
VIB4920 demonstrates a good safety profile in humans

| | | VIB4920 | VIB40920 | VIB4920 | VIB4920 | VIB4920 | VIB4920 | VIB4920 | VIB4920 |
|---|---|---|---|---|---|---|---|---|---|
| TEAE (Preferred | Placebo | 3 mg | 10 mg | 30 mg | 100 mg | 300 mg | 1000 mg | 3000 mg | Total |
| term), n (%) | N = 15 | N = 2 | N = 2 | N = 8 | N = 8 | N = 8 | N = 8 | N = 8 | N = 44 |
| Nasopharyngitis | 1 (8.3) | 0 | 1 (50.0) | 0 | 5 (62.5) | 1 (12.5) | 1 (12.5) | 2 (25.0) | 10 (22.7) |
| Headache | 4 (33.3) | 0 | 1 (50.0) | 2 (25.0) | 0 | 1 (12.5) | 1 (12.5) | 3 (37.5) | 8 (18.2) |
| Diarrhea | 0 | 0 | 1 (50.0) | 0 | 0 | 1 (12.5) | 2 (25.0) | 1 (12.5) | 5 (11.4) |
| Oropharyngeal pain | 1 (8.3) | 0 | 1 (50.0) | 1 (12.5) | 1 (12.5) | 0 | 0 | 1 (12.5) | 4 (9.1) |
| Cough | 1 (8.3) | 0 | 0 | 1 (12.5) | 1 (12.5) | 0 | 1 (12.5) | 0 | 3 (6.8) |
| Nasal Congestion | 1 (8.3) | 0 | 1 (50.0) | 0 | 1 (12.5) | 0 | 0 | 1 (12 5) | 3 (6.8) |
| Rhinorrhea | 1 (8.3) | 0 | 0 | 1 (12.5) | 0 | 0 | 1 (12.5) | 1 (12.5) | 3 (6.8) |
| Abdominal Pain | 0 | 0 | 0 | 0 | 0 | 1 (12.5) | 0 | 1 (12.5) | 2 (4.5) |
| Ear discomfort | 0 | 0 | 0 | 0 | 2 (25.0) | 0 | 0 | 0 | 2 (4.5) |
| Oral herpes | 1 (8.3) | 0 | 0 | 0 | 0 | 0 | 0 | 2 (25.0) | 2 (4.5) |
| Vomiting | 1 (8.3) | 0 | 1 (50.0) | 0 | 0 | 1 (12.5) | 0 | 0 | 2 (4.5) |

Most common TEAEs occurring in at least 2 subjects in a Ph1a study of healthy volunteers

Importantly, the overall percentage of subjects with one or more investigational product-related TEAE was comparable between the total VIB4920 group (40.9%) and placebo (33.3%). Additionally, there were no infusion-related reactions, severe infections or deaths, and only a single TESAE reported, a tibia fracture in the placebo group. Notably, in this Ph1a study, no clinically significant coagulation or platelet function abnormalities were observed following treatment with VIB4920.

### Example 6—VIB4920 Demonstrates a Favorable PK/PD Profile in Healthy Volunteers

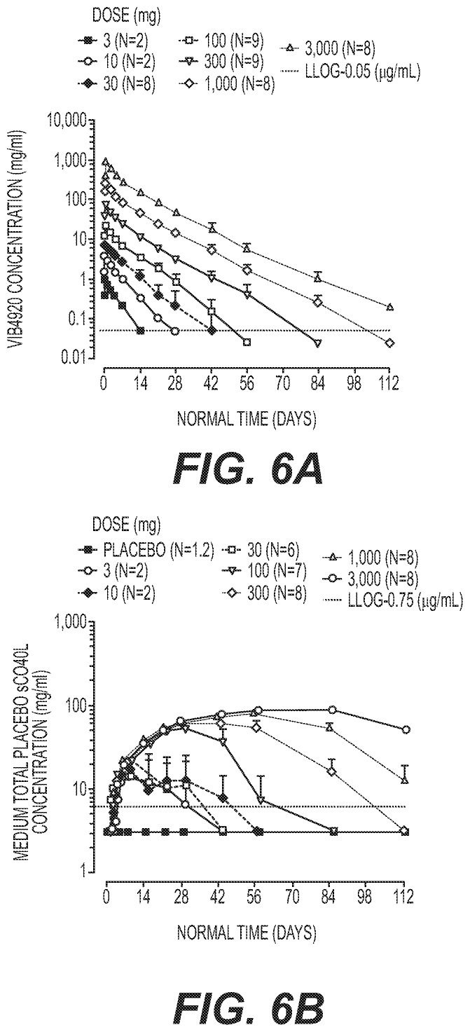

In addition to evaluating the safety profile of VIB4920, pharmacokinetic (PK) and pharmacodynamic (PD) endpoints were also evaluated in the Ph1a study. The PK profile of VIB4920 following a single intravenous dose of 3-3000 mg was linear with increasing exposure in a dose-proportional manner (FIG. 6A). The mean terminal half-life of the molecule was 8 days with a half-life up to 10.1+/−1.87 days at the highest dose.

CD40L is a transmembrane protein; however, it can be cleaved and shed by both activated T cells and platelets. Soluble CD40L (sCD40L) is an 18-KDa trimer that is detected at low levels in healthy donors and increased in the circulation of patients with autoimmune disease (36, 37). Measurement of sCD40L levels following VIB4920 administration represents a potential measure of target engagement, as sCD40L bound to VIB4920 could be retained and accumulate in circulation. As expected, there was a dose dependent increase in total sCD40L in the plasma following administration of VIB4920 (FIG. 6B), suggesting target engagement. The time to reach the maximum total sCD40L in the plasma increased from 11.5 to 84 days as the dose increased from 3 mg to 3000 mg, indicating target engagement was maintained for a longer duration in the highest dose group.

### Example 7—Reduced ADAs were Observed in Healthy Subjects Receiving Higher VIB4920 Doses

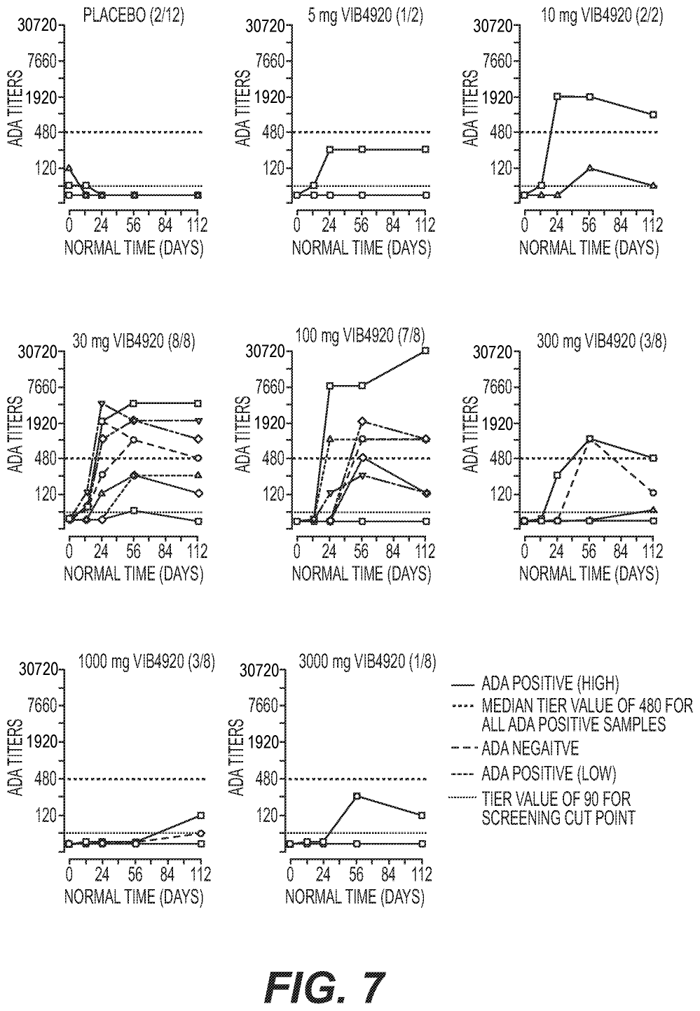

Biological drugs are by nature highly specific/selective; however, they are complex molecules capable of eliciting an immune response. Anti-drug antibodies (ADAs) are a measure of the immunogenicity of a therapeutic. In healthy volunteers, ADAs were detected in the clear majority of patients receiving low doses of VIB4920 (FIG. 7). More specifically, 18 of 20 subjects in the 3-100 mg dose range had detectable ADAs, with 10 of those individuals exhibiting high ADA titers (greater than the median titer value of 480). In contrast, the frequency of ADAs was significantly reduced at higher dose levels of VIB4920 (FIG. 7), with only 1 of 8 subjects in the 3000 mg dose group generating detectable anti-drug titers. The reduction in ADA frequency observed at high doses of VIB4920 supports the immunomodulatory capacity of the molecule. Additionally, low percentages and titers of ADAs may translate to a better tolerated, more efficacious therapeutic.

### Example 8—VIB4920 Inhibits T-Cell Dependent Antibody Response in Healthy Volunteers

VIB4920 was further evaluated for its ability to influence humoral immune responses in healthy subjects. This evaluation was performed by determining VIB4920's effect on a T-cell dependent antibody response (TDAR), which was induced by immunization with keyhole limpet hemocyanin (KLH). Healthy subjects in all treatment groups received two subcutaneous KLH immunizations: (first) at 14 days prior to dosing with either VIB4920 or placebo and (second) at 15 days post dosing (FIG. 5B). Both IgM and IgG antibodies generated against KLH, were monitored out to day 113.

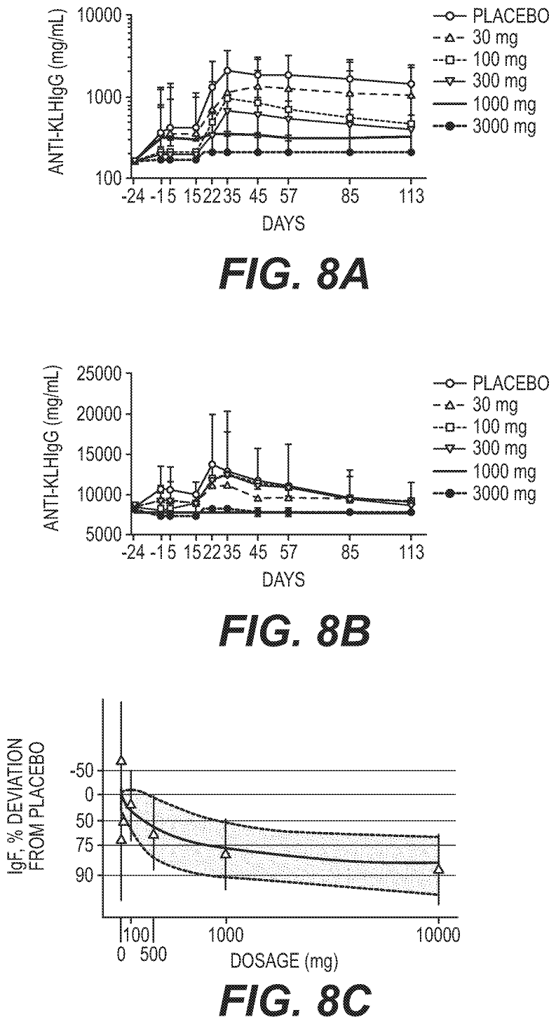

TDAR followed an expected trend in placebo-treated subjects, i.e., a trend including a sharp increase in anti-KLH IgG titers on day 22, (one week following the secondary immunization), peak levels of IgG observed on day 29, and then a decline in KLH specific IgG antibodies out to the end of the monitoring period (FIG. 8A). Furthermore, and consistent with previous reports, the secondary anti-KLH response in the placebo-treated group was dominated by IgG, with overall a much more modest increase in KLH-specific IgM detected following re-challenge (FIG. 8B) (38-41).

As anticipated, healthy volunteers treated with VIB4920 at low doses had anti-KLH titers close to that of the placebo-treated group. In contrast, healthy volunteers treated with VIB4920 at higher doses exhibited a significantly reduced secondary response to KLH, such that on day 43 there was statistically significant reduction in anti-KLH IgG starting with the 300 mg dose (p=0.035) and increasing with the 1,000 mg (p=0.002) and 3,000 mg (p<0.001) doses. Of note, IgG to KLH was reduced by 78% and 86% compared to placebo at day 43 in the 1000 mg and 3000 mg cohorts, respectively (FIG. 8C). In the highest dose group, 7 of 8 subjects had undetectable titers of anti-KLH-IgG at day 43, suggesting near complete suppression of the humoral immune response by VIB4920.

### Example 9—VIB4920 Immunosuppression is Mediated Through Inhibition of B Cell Proliferation and Plasma Cell Responses

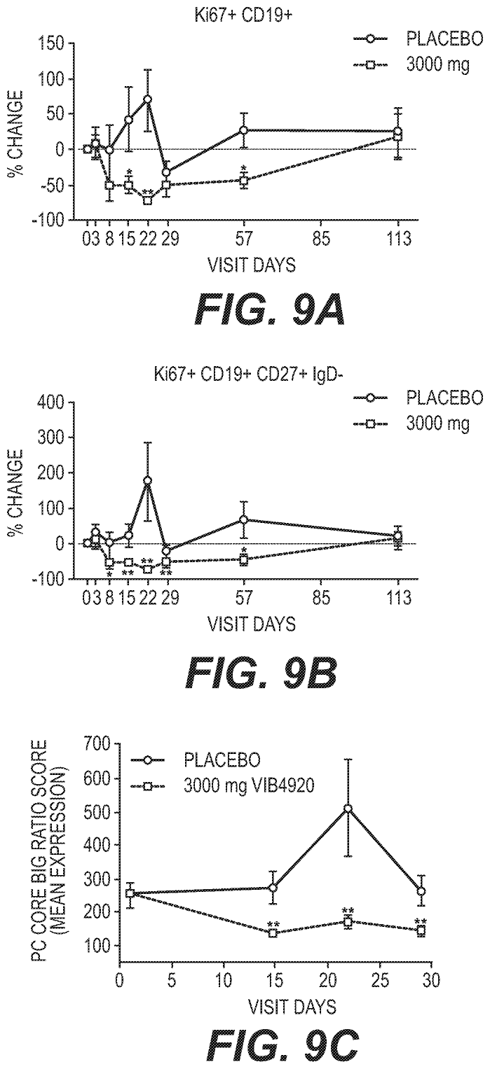

The mechanism by which VIB4920 suppresses secondary immune responses was better defined by collecting peripheral blood from subjects before and after immunization, and characterizing circulating lymphocyte subsets by flow cytometry. In placebo-treated healthy subjects, secondary immunization induced B cell proliferation, which was indicated by detection of an increase in the frequency of Ki67+CD19+ B cells in the circulation on visit day 22, i.e., 7 days post re-challenge (FIG. 9A).

In subjects that received high dose VIB4920, and prior to the re-challenge, the baseline frequency of proliferating B cells was reduced compared to the placebo-treated group. This is consistent with the proposed mechanism of action of the molecule. Furthermore, in the cohorts receiving high dose VIB4920, the B cell proliferative response following immunization was significantly impaired, demonstrated by the lack of increase in Ki67+ B cells. See the 3000 mg cohort one week post-challenge (FIG. 9A). Further phenotyping revealed that the greatest impact of VIB4920 on proliferating B cells was noted within the IgD-CD27+ isotype switched memory population (FIG. 9B). These data are consistent with the TDAR results which demonstrate the suppressive impact of VIB4920 on IgG production in response to secondary challenge.

Changes in gene expression were also monitored in peripheral blood of placebo or VIB4920 treated subjects prior to and following secondary immunization with KLH. Specifically, a plasma cell (PC) gene signature, an accurate and robust signature capable of detecting even subtle changes in circulating PC frequency (42), was used to ascertain certain changes in gene expression. Consistent with the TDAR results, immunization induced a dramatic increase in the PC gene signature score in placebo-treated subjects' whole blood at one week following re-challenge, which returned to baseline by two weeks (FIG. 9C). In the highest VIB4920 dose cohort (3000 mg), the PC gene signature score in peripheral blood was significantly reduced compared to placebo-treated subjects prior to re-challenge with KLH (FIG. 9C). Importantly, there was no increase in PC gene signature score following re-immunization in volunteers receiving high dose VIB4920. These data highlight the mechanism of action of VIB4920 and demonstrate its potent ability to suppress B cell and PC responses.

### Example 10—Multiple Dose Administration of VIB4920 in RA Patients is Safe and Well Tolerated

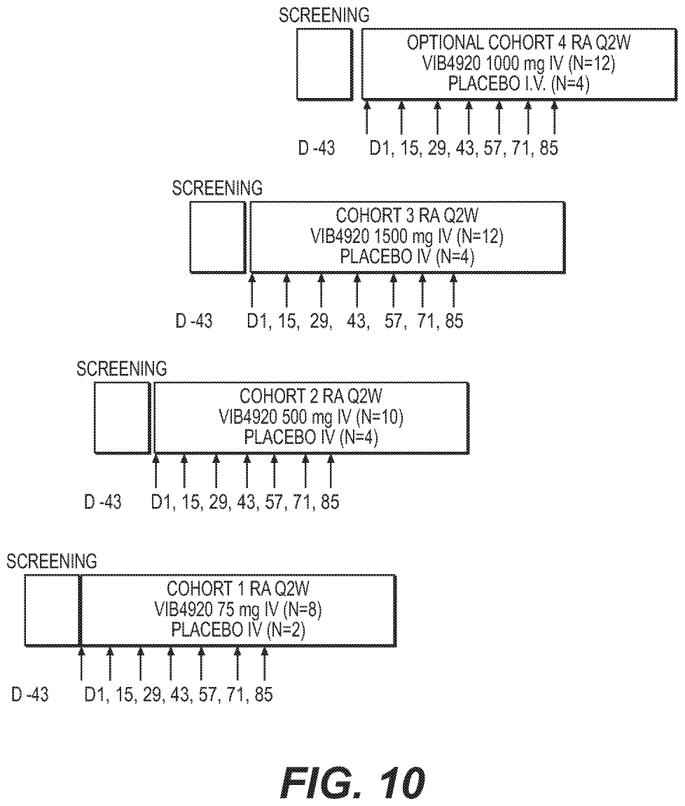

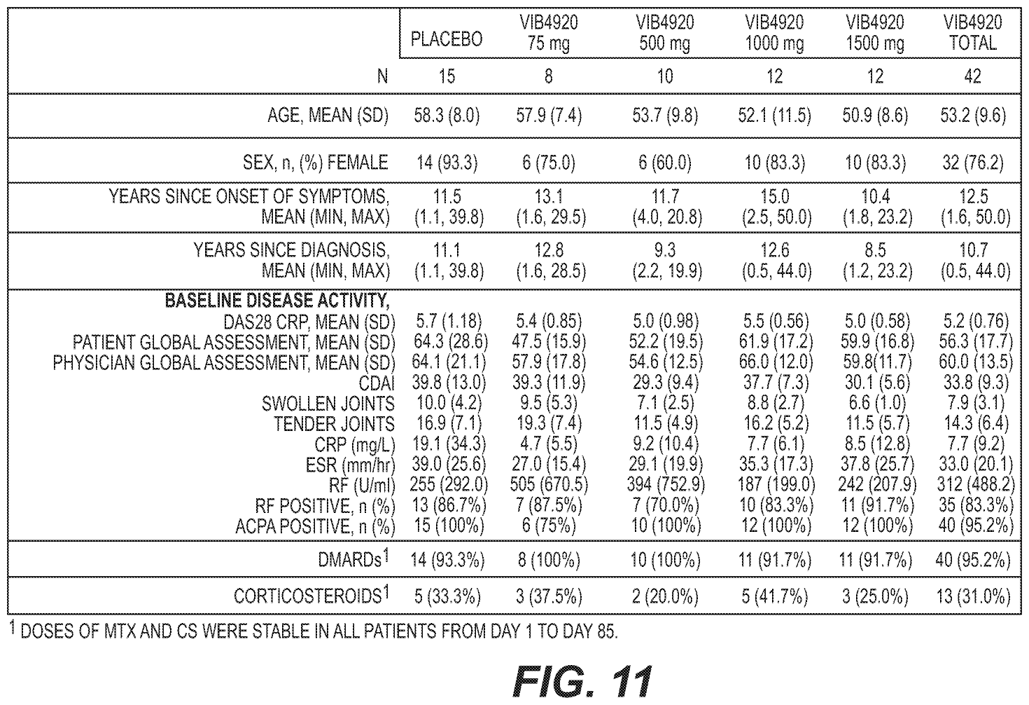

Having established VIB4920 has an acceptable safety profile and demonstrates proof-of-mechanism in healthy volunteers, a multiple ascending dose, proof of concept Ph1b clinical study was conducted in adult patients with moderate to severe active RA. RA patients were treated with VIB4920 (75 mg, n=8; 500 mg, n=10; 1000 mg, n=12; or 1500 mg, n=12) or placebo (n=15), administered by intravenous (i.v.) infusion, every other week for 12 weeks (FIG. 10). The patients were then observed for an additional 12 weeks post-treatment. Key endpoints measured at week 12 included safety, tolerability, PK parameters, ADAs, and change in disease activity (DAS28-CRP) as well as additional biomarkers such as RF autoantibodies, serum C-reactive protein (CRP), and Vectra-DA score. Fifty-three patients completed 12 weeks of treatment; two patients (one in VIB4920 75 mg group and one in VIB4920 1500 mg group) discontinued treatment due to adverse events, one patient in the placebo group withdrew informed consent and one patient in the VIB4920 75 mg group was lost to follow up. The main demographic and clinical characteristics of the study population at baseline are presented in FIG. 11.

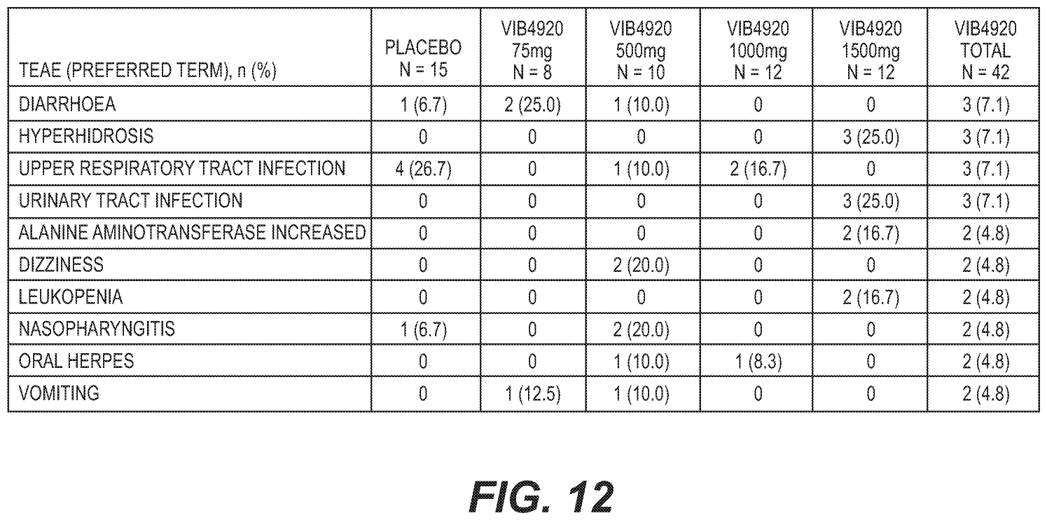

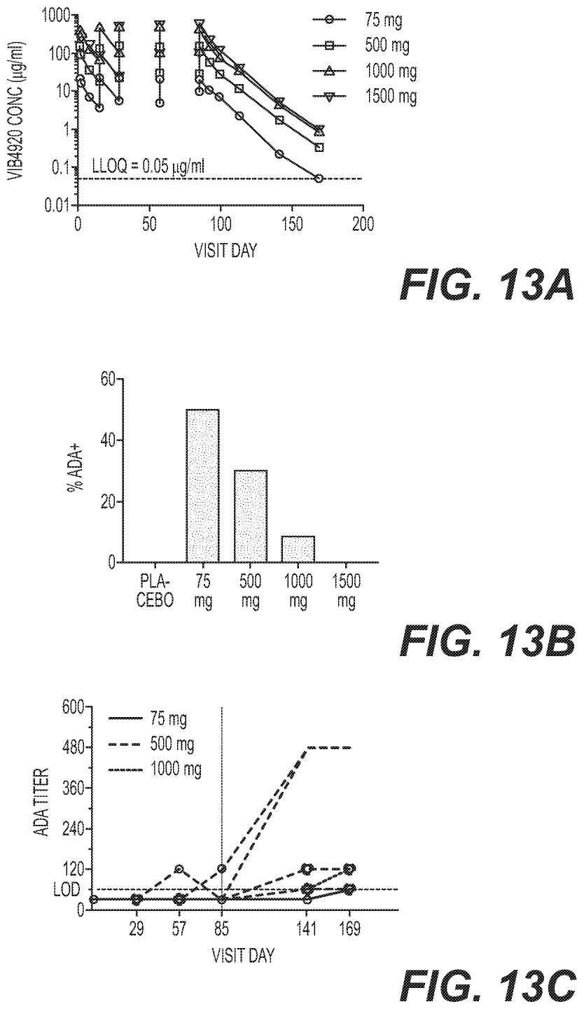

Overall, VIB4920 was generally safe and well tolerated with a balanced distribution of TEAEs observed between placebo and the four active dose groups. The most common TEAEs reported were diarrhea, hyperhidrosis, upper respiratory tract infection and urinary tract infection, each occurring in 3 patients (7.1%). See FIG. 12. No thrombotic adverse events or clinically significant coagulation abnormalities were noted. One adverse event (preferred term ‘encephalitis) was reported as serious and life-threatening, occurring in the 1500 mg dose group after 6 doses of study drug. No etiological infectious agent was identified and several months after discontinuing VIB4920 similar symptoms recurred and patient was subsequently diagnosed with metastatic melanoma of the brain.

### Example 11—VIB4920 Demonstrated a Linear PK Profile and Dose-Dependently Reduced ADAs in RA Patients

ADAs were observed in RA patients receiving low dose VIB4920, similar to the healthy Ph1a study volunteers receiving low dose VIB4920. Three of 8 (37.5%) of the RA patients receiving 75 mg VIB4920, and 3 of 10 (30%) of the RA patients receiving 500 mg VIB4920 developed ADAs (FIG. 13B). In the 75 mg VIB4920 dose group, 2 out of 8 subjects developed detectable ADAs during the treatment phase; all 3 subjects in 500 mg VIB4920 treatment group developed detectable ADAs post-treatment (FIG. 13C). No ADAs were detected in the 1000 mg dose group during the treatment period; one subject had detectable ADA after the treatment phase. No ADAs were detected in the 1500 mg dose group (FIG. 13B), suggesting that VIB4920 effectively suppresses the ADA response at higher doses.

### Example 12—VIB4920 Reduces Disease Activity in Patients with RA

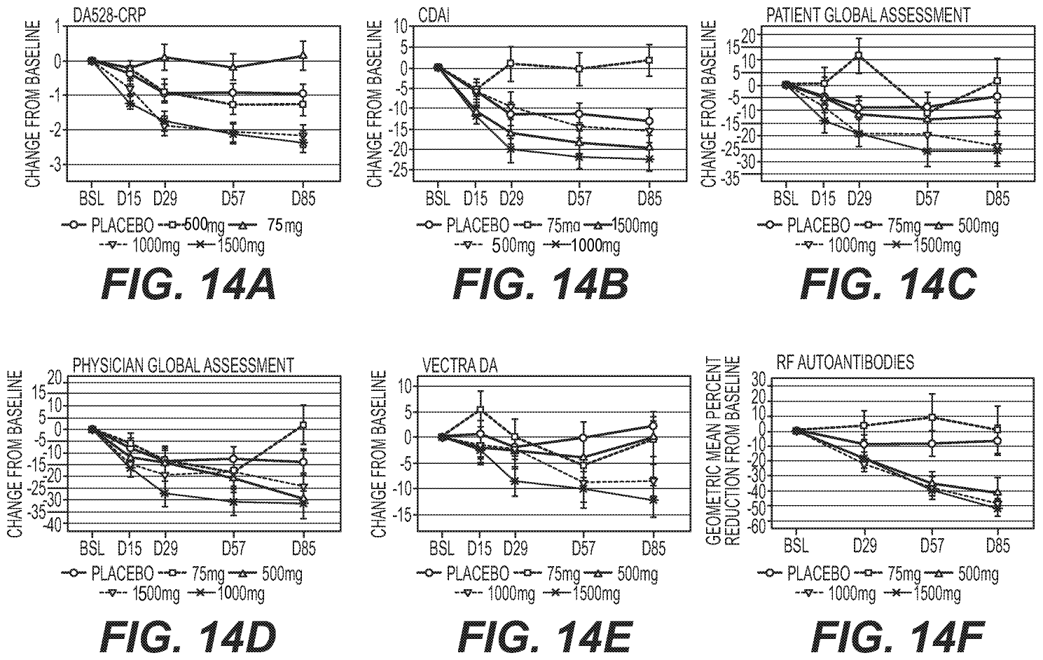

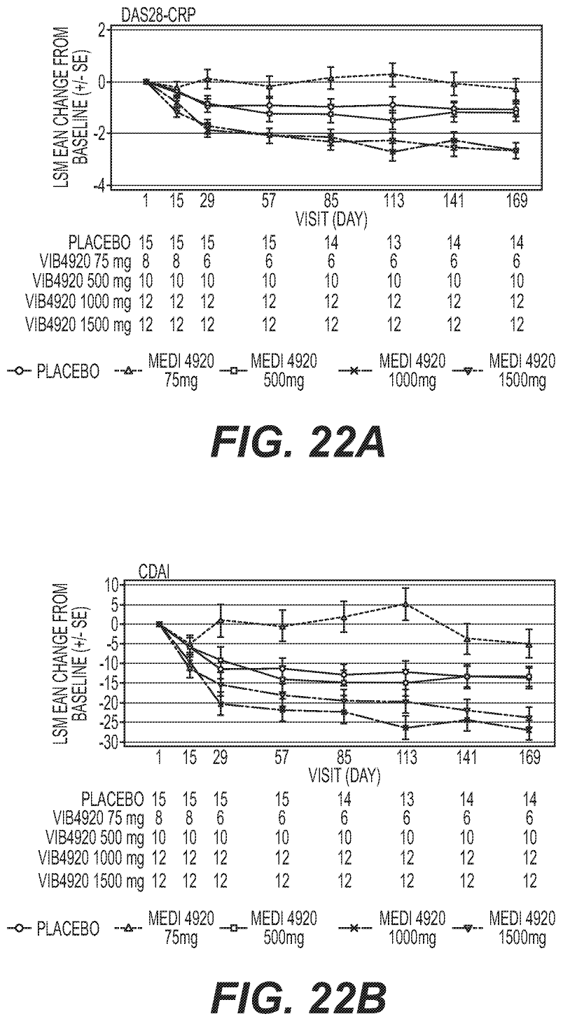

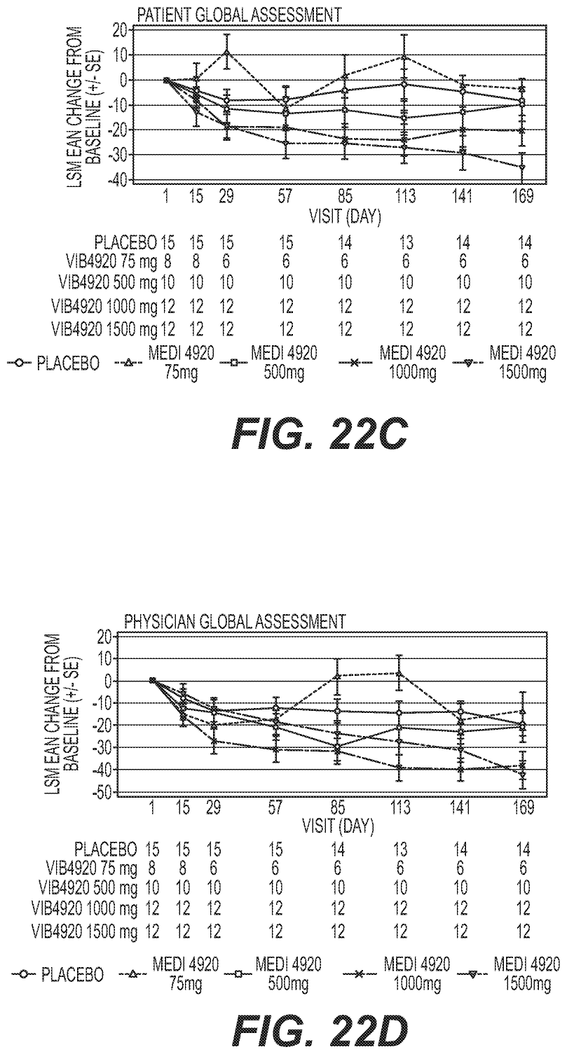

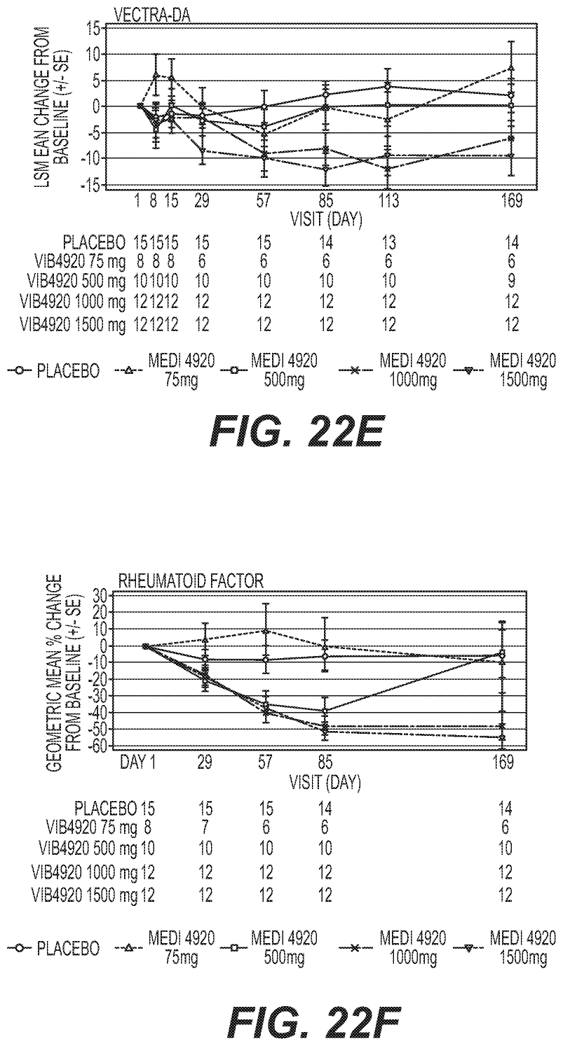

DAS-28/CRP scores were determined to ascertain whether VIB4920 reduced disease activity in the RA patients of the phase 1b clinical trial. The DAS-28/CRP score is a composite clinical disease activity score, used in RA, that takes into account: number of swollen joints, number of tender joints, CRP levels, and a patient global health assessment. VIB4920 significantly reduced disease activity quantified by the DAS28-CRP score in RA patients at higher doses (FIG. 14A and FIG. 22A). At week 12 post-treatment initiation, the adjusted mean change from baseline of DAS28-CRP (SE) was: −2.3 (0.3) in the VIB4920 1500 mg group, −2.2 (0.3) in the VIB4920 1000 mg group, −1.2 (0.3) in the VIB4920 500 mg group, 0.1 (0.4) in the VIB4920 75 mg group and −1.0 (0.3) in the placebo group (FIG. 14A). Surprisingly, this observed DAS28-CRP score reduction in RA patients was maintained for at least an additional 12 weeks after administration of the last VIB4920 dose. (See FIG. 22A, in particular, visit days 113, 141, and 169). The effect of VIB4920 on DAS28-CRP was rapid, with reductions in score evident by Day 15, which was after only a single dose of drug.

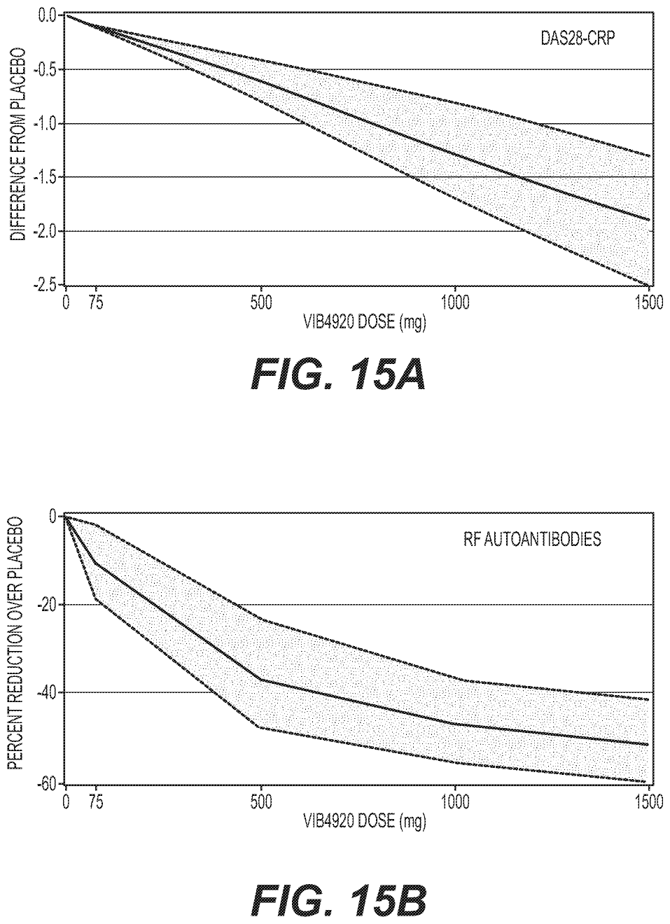

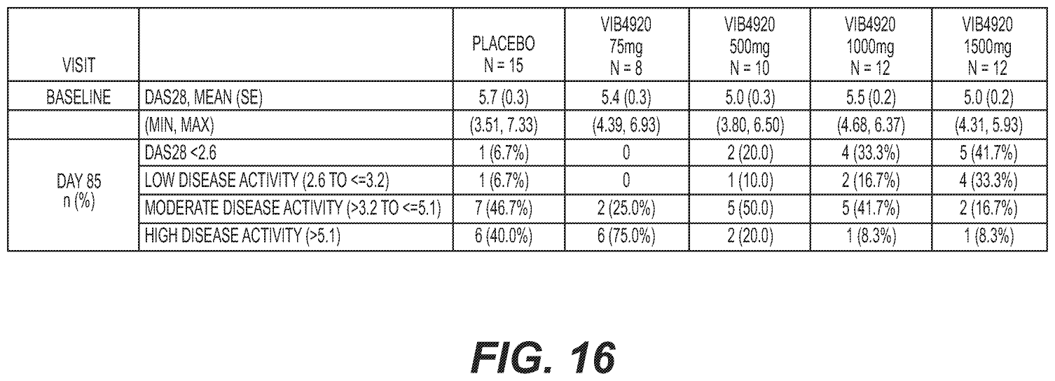

Moreover, the reduction of disease activity at the two highest doses of VIB4920, as compared with placebo, was both clinically and statistically meaningful; the adjusted mean difference at Week 12 (SE) for the VIB4920 1500 mg group was −1.4 (0.4) and for the VIB4920 1000 mg group was −1.2 (0.4), p-values of 0.002 and 0.006, respectively. Using a linear dose response model, a statistically significant dose-response was demonstrated for DAS28CRP (p<0.001). The significant result was mainly driven by the 1000 mg and 1500 mg treatment groups; the 500 mg and 75 mg showed little to no benefit over placebo (FIG. 15A). In terms of individual clinical response, 75% of patients in the 1500 mg group and 50% of patients in 1000 mg dose group achieved a DAS28-CRP of score of 3.2 or less at week 12, indicating they were in low disease activity or clinical remission at the primary endpoint. See FIG. 16.

### Example 13—VIB4920 Reduces Immunological and Inflammatory Biomarkers in Patients with RA

The effect of VIB4920 on immunological and inflammatory biomarkers was determined using the Vectra DA blood test. The Vectra DA test is a commercially available and validated test that measures 12 biomarkers (adhesion molecules, growth factors, cytokines, matrix metalloproteinases, skeletal proteins, hormones and acute phase proteins) of disease activity and combines them into a single score for assessment of the key mechanisms and pathways that drive RA disease activity. VIB4920, at doses of 1500 mg and 1000 mg, significantly reduced the Vectra DA multi-biomarker score both during the 12 week time period in which VIB4920 was administered every other week (FIG. 14E) and during the 12 week observation period during which VIB4920 was no longer administered (FIG. 22E). The adjusted mean difference for VIB4920 at the 1500 mg dose vs placebo (Week 12) was −14.4 (−21.5, −7.2), p=0.001, and the adjusted mean difference for VIB4920 at the 1000 mg dose vs placebo (Week 12) was −10.3 (−17.4, −3.3), p=0.018 (FIG. 14E).

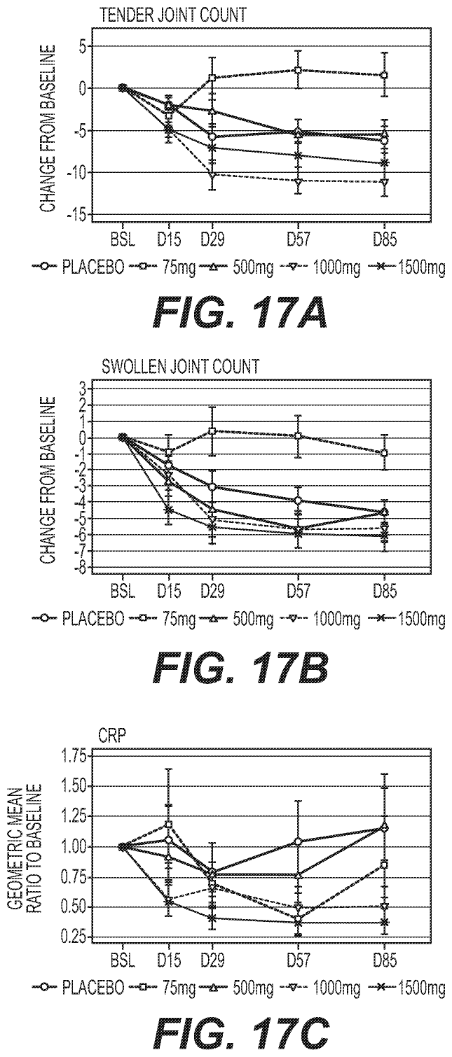

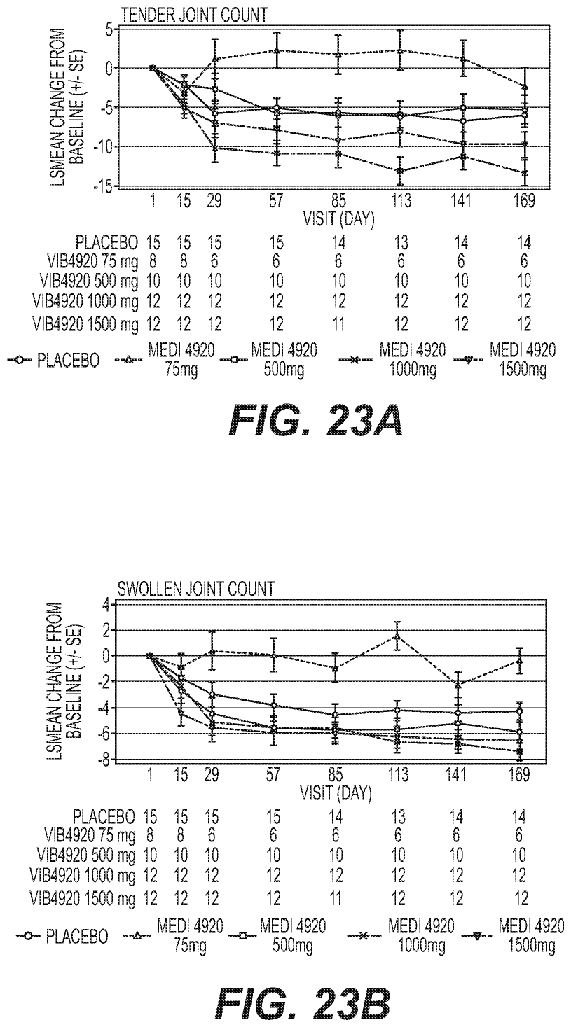

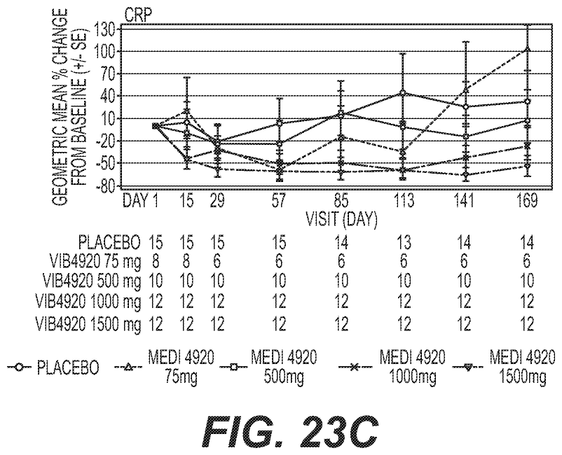

The efficacy results were highly consistent across other endpoints evaluated in this trial (including Clinical Disease Activity Index—CDAI, tender and swollen joint counts, patient's and physician's global assessment, and serum CRP level) supporting 1000 and 1500 mg as clinically efficacious doses in this study (FIG. 14B-14D and FIG. 17A-17C; See also FIG. 22B-22D and FIG. 23A-23C).

### Example 14—VIB4920 Significantly Reduces Rheumatoid Factor Autoantibodies of RA Subjects

Rheumatoid factor autoantibodies (RFs) are a family of autoantibodies produced against the Fc portion of IgG. They are elevated in RA and are associated with a poor prognosis. Given the mechanism of action of VIB4920, its impact on autoantibody titers in RA subjects was assessed. Notably, VIB4920 significantly reduced RF titers at the 500, 1000 and 1500 mg dose levels (FIG. 14F) during the 12 week every-other-week treatment period. Furthermore, and surprisingly, the reduced RF titers at the 1000 and 1500 mg dose levels were maintained throughout the 12 week observation period following administration of the last VIB4920 dose (FIG. 22F). Reductions in the RF titers from baseline were evident in response to VIB4920 as early as day 29, with high dose VIB4920 reducing RF titers by approximately 50% by day 85. Using an Emax model, VIB4920 demonstrates a statistically significant dose response in terms of reduction of RF titers from baseline (p<0.001) (FIG. 15B).

### Example 15—Methods

NF-kB Reporter Assay.

HEK293 cells expressing an NF-kB luciferase reporter (Panomics) were engineered to stably express human full-length CD40R. Cells were seeded at a density of 5×10⁴ cells/well in a 96-well poly-D-Lysine coated plates (BD Biosciences) and stimulated with megaCD40L recombinant protein (1.5 ug/ml, Enzo Biosciences) or CD40L overexpressing D1.1 Jurkat subclone (ATCC) cells for 16-24 hours in the presence or absence of control or CD40L specific Tn3s at indicated concentrations. Luminescence was detected using the Bright-Glo Luciferase Assay System (Promega) on a SpectraMax M5 plate reader (Molecular Devices).

CD86 Upregulation Assay

Human blood was collected from healthy donors following informed consent as approved by MedImmune's Institutional Review Board. Peripheral blood mononuclear cells were isolated from CPT tubes (BD Biosciences) following centrifugation. PBMCs (2.5-5.0×10⁵ cells/well) were stimulated in a 96-well round bottom plate with recombinant megaCD40L (100 ng/ml, Enzo Biosciences) for 16-18 hours in the presence of CD40L-specific Tn3s or mAb (clone 5c8) as indicated. Flow cytometry was used to evaluate CD86 expression on CD19+ B cells. The following antibodies were used: CD86 (clone 2331, BD Pharmingen) CD19 (clone HIB19, BD Pharmingen).

Human B Cell Assay

PBMCs were isolated. Total B cells were negatively selected using MACS cell separation technology (Miltenyi Biotec), which routinely yielded greater than 95% purity. Purified peripheral blood B cells were cultured at a density of 0.5 to 1.0×10⁵ B cells per well in 96-well round-bottom plates in a final volume of 150 μl complete medium. Culture medium for B cell experiments was RPMI 1640 (Invitrogen) supplemented with 10% FCS, penicillin-streptomycin (100 units/ml penicillin, 100 μg/ml streptomycin), 2-mercaptoethanol (55 μM), L-glutamine (2 mM), and HEPES (5 mM). At initiation of culture, B cells were stimulated with a combination of IL-21 (33 ng/ml, PeproTech Inc.) and megaCD40L (1.5 nM, Enzo Biosciences) with or without anti-IgM F(ab')₂ (5.0 μg/ml, Jackson ImmunoResearch Laboratories). B cell expansion was quantified by measuring ATP on day 3 or day 4 of culture using the Cell Titer-Glo Luminescent Assay (Promega), according to the manufacturer's instructions. PC differentiation was quantified on day 7 by flow cytometry. Cells were acquired for a fixed amount of time and PCs were defined as CD19⁺IgD⁻CD38^{hi} cells.

Murine SRBC Immunization Model

Balb/c mice (Jackson Laboratories) were immunized on day 0 with 0.2 ml of SRBC (Colorado Serum Company), by intraperitoneal injection after withdrawing directly from bottle. Control (30 mg/kg) or CD40L-specific Tn3s (up to 30 mg/kg, as indicated) were administered daily from days 9-13 (intravenously). The frequency of germinal center B cells in the spleen was quantified on day 14 by flow cytometry. GC B cells were defined as CD19B220⁺Fas⁺PNA⁺ B cells.

Platelet Aggregation Assay

Human blood was collected from healthy donors into ACD Solution B tubes containing citric acid, dextrose and sodium. Following centrifugation, two thirds of the platelet rich plasma was transferred into a polypropylene tube and incubated for 10 minutes with apyrase (2 U/ml) to prevent platelet activation during processing. Platelets were pelleted and resuspended in modified Tyrode's buffer (137 mM NaCl, 2.7 mM KCl, 1 mM MgCl2, 5.6 mM dextrose, 3.3 mM NaH2PO4, 20 mM HEPES, 0.1% BSA, and pH 7.4).

Immune complex (IC) was generated by mixing the mAb (h5c8 or negative ctrl antibody) or anti-CD40L Tn3 with hCD40L (293 Cell Source) for 5 minutes at room temperature. In some experiments platelets were pre-incubated with anti-CD32a antibody (IV.3) for five minutes prior to addition of IC. Platelet aggregation assay was performed according to manufacturer's instructions with stirring at 37° C. in a four-channel optical platelet aggregometer (model 700, Chrono-Log, Havertown, Pa.). Light transmission was monitored for 12-20 minutes after mixing washed platelets with agonists.

Ph1a Subjects and Study Design

A Phase I, randomized, blinded, placebo-controlled study was conducted in healthy adults aged 18-49, including females of non-childbearing potential (NCT02151110). Subjects were randomized into seven dose cohorts (3, 10, 30, 100, 300, 1000, or 3000 mg) and were dosed sequentially based on the study protocol and recommendations from a Dose Escalation Committee (DEC) that reviewed the safety and tolerability data from the current dose cohort as well as the accumulated data from the previous dose cohorts. TDAR was induced in subjects by administering two separate immunizations of 1 mg KLH subcutaneously. The first KLH immunization was administered during the screening period, 14 days prior to dosing with either VIB4920 or placebo, and the second KLH immunization was administered on Day 15 after dosing with either VIB4920 or placebo. Three interim analyses were conducted per the protocol when all subjects in Cohort 5 (300 mg), Cohort 6 (1000 mg), and Cohort 7 (3000 mg) completed Day 43, respectively.

PK Assay for VIB4920

VIB4920 in human K2EDTA plasma was measured using a validated sandwich ELSA method in which wash steps with 1×PBS/0.1% Tween 20 (PBST) followed each incubation to remove unbound components. Briefly, Nunc microtiter plates were coated overnight at 2-8° C. with 1 μg/mL anti-VIB4920 mouse monoclonal antibody (MedImmune). Standards, quality controls (QCs) and samples containing VIB4920 were diluted to the method minimum required dilution (MRD) of 1:50 in 0.5% bovine serum albumin (BSA)/PBST prior to plate addition. Following a 2-hour incubation, 1 μg/mL anti-VIB4920 rat antibody (MedImmune) that had been labeled with biotin was added to the plate and incubated 1 hour. The binding complex was visualized with successive incubations of streptavidin-linked horseradish peroxidase (HRP, GE Healthcare) and SureBlue™ tetramethylbenzidine (TMB) peroxidase substrate (KPL, Inc.). Color development was stopped with 0.2 M sulfuric acid prior to analysis at 450 nm on a microplate reader. The quantitative range was 0.05 to 1.60 μg/mL; samples measuring above the quantitative range were diluted with pooled K2EDTA plasma to bring the concentration within the measurable range of the method.

Quantitation of sCD40L

Plasma samples were collected for measurement of sCD40L concentrations during the screening period and on Days 1, 2, 3, 5, 8, 15, 22, 29, 43, 57, 85, and 113. Total soluble CD40L (free sCD40L and sCD40L bound to VIB4920) in human K2EDTA plasma was measured using a human sCD40L Platinum ELISA kit (eBioscience) that had been modified to meet program needs and qualified to ensure accuracy and precision. Briefly, standards, QCs and samples containing sCD40L were diluted to the method MRD of 1:50 in assay diluent containing 0.5% BSA/PBST and VIB4920 to ensure comparable and consistent results. Wash steps with PBST followed each incubation to remove unbound components. The diluted samples were added to a plate pre-coated with anti-sCD40L antibody, and incubated for 1.5 hours. HRP-conjugated anti-human sCD40L was then added to bind to sCD40L captured by the coat antibody. The binding complex was visualized with successive additions of TMB peroxidase substrate and stop solution (phosphoric acid) prior to analysis at 450 and 540 nm on a microplate reader. The quantitative range was 6.25 to 400.00 ng/mL; samples measuring above the quantitative range were diluted with pooled K2EDTA plasma to bring the concentration within the measurable range of the method.

Measurement of ADA

The presence of ADAs to VIB4920 in human K2EDTA plasma was determined using a validated sandwich ELISA method in which wash steps with PBST followed each incubation to remove unbound components. Briefly, QCs and samples were diluted to the method MRD of 1:60 in assay diluent containing 0.5% BSA/PBST, added to a washed Pierce™ Protein G coated plate (ThermoFisher), and incubated 2 hours. Overnight incubation of 1 μg/mL Biotin-labeled VIB4920 prepared in assay diluent, specifically detected ADA to VIB4920. The binding complex was visualized with successive incubations of streptavidin-linked HRP (GE Healthcare) and SureBlue™ TMB peroxidase substrate (KPL, Inc.). Color development was stopped with 0.2 M sulfuric acid prior to analysis at 450 nm on a microplate reader. Each sample was subject to a three-tier process where the sample response was first compared to a statistically determined cutoff OD value, at or above which a sample was considered potentially positive, and below which the sample was determined negative for ADA. The potentially positive samples were subjected to a second, competition evaluation in the presence of excel VIB4920; samples with a percent inhibition at or above the statistically determined confirmatory cut point were defined as confirmed positive and taken into a titer evaluation. Samples below the confirmatory cut point were considered negative for ADA to VIB4920. Titered samples were serially diluted in pooled human K2EDTA plasma to below the screening cutoff, and the titer result reported as the reciprocal of the highest dilution at which the sample measured positive before measuring negative.

Assessment of Anti-KLH Antibodies

Anti-keyhole limpet hemocyanin (KLH) IgG antibody in human serum was measured using a validated sandwich ELISA method in which wash steps with PBST followed each incubation to remove unbound components; 100 μL volume/well was used for all steps. Briefly, nunc microtiter plates were coated overnight at 2-8° C. with 3 μg/mL KLH (Immucothel, biosyn Arzneimittel GmbH) prepared in 1×PBS, pH 7.2. Standards and QCs, comprised of a mixture of nine monoclonal anti-KLH IgG antibodies of varying isotype and affinity (AstraZeneca), and samples containing anti-KLH antibodies were diluted to the method MRD of 1:250 in 0.5% BSA/PBST prior to plate addition. Following a 2-hour incubation, HRP-conjugated mouse anti-human IgG (Invitrogen) was added to the plate and incubated 1 hour to specifically detect anti-KLH IgG antibodies. The binding complex was visualized with successive additions of TMB peroxidase substrate and stop solution (0.2 M sulfuric acid) prior to analysis at 450 on a microplate reader. The quantitative range was 163.30 to 10000.00 ng/mL; samples measuring above the quantitative range were diluted with serum to bring the concentration within the measurable range of the method.

Flow Cytometry in pH1a

Blood was collected in Cytochex BCT tubes (Streck), shipped to Covance Central Laboratory Services (Indianapolis, Ind.), and tested by flow cytometry using a validated method. Briefly cells were stained with fluorochrome labelled antibodies to CD45 (clone HI30), CD19 (Clone HIB19), IgD (Clone IA6-2), CD27 (Clone M-T271), and CD38 (Clone HIT2, all BD) to identify B cell populations. Cells were subsequently treated with FACSPerm2 (Becton Dickenson) and stained for intracellular Ki67 (clone KI67, Biolegend) expression to measure proliferating cells.

PC Signature

PC gene signature was determined as previously described (Streicher 2014). Briefly, Total RNA was extracted from PAXgene blood tubes using a PAXgene Blood RNA kit (Qiagen). For TaqMan qPCR, cDNA was generated using a SuperScript III First-Strand Synthesis SuperMix kit (Life Technologies) and random primers. Samples were prepared using a TaqMan Pre-Amp Master Mix kit and analyzed with a BioMark Real-Time PCR System. We calculated ΔΔCt values using the mean of 2 reference genes ((3-actin and GAPDH) and each patient's baseline expression level as controls. Fold change values were determined by calculating 2-ΔΔCt.

Ph1b Patients and Study Design

A Phase 1b randomized, blinded, placebo-controlled study was conducted in patients aged 18-70 years old diagnosed with RA according to EULAR/ACR criteria (Aletaha et al. 2010) for at least 6 months before entering the study. Subjects had moderate to severe activity as defined by a DAS28-CRP score of at least 3.2 at screening and at least 4 swollen and 4 tender joints at screening and randomization. Patients were positive for either rheumatoid factor (RF-IgM≥14 units/mL) or anti-citrullinated peptide antibodies (ACPA) at screening. Patients received methotrexate (MTX) at a dose of 7.5-25 mg per week or, in case of MTX intolerance, a different conventional DMARD, started at least 12 weeks and at a stable dose for at least 6 weeks prior to screening. Previous treatment with biological agents (except Rituximab or other B-cell depletive agents) given for RA was accepted provided proper washout was done before randomization in our study. Patients were treated with placebo (n=15) or VIB4920 (75 mg, n=8; 500 mg n=10; 1000 mg n=12; or 1500 mg n=12) given by i.v. infusion every other week for 12 weeks followed by 12 weeks of post-treatment observation. Measurements for VECTRA-DA score were performed by Crescendo Bioscience (San Francisco, Calif.) and RF autoantibody measurements were performed by Covance Central Laboratories Services (Princeton, N.J.).

Statistical Analysis

Two-tailed unpaired Students t-tests were used to evaluate the impact of treatment on primary human B cell expansion, plasma differentiation and the GC B cell response in the SRBC model. Mann-Whitney U test was used to compare VIB4920 versus placebo at multiple time points for gene signature score (FIG. 9C). Statistical tests and plots were performed using Graphpad Prism software. The dose response for change from baseline in DAS28-CRP and RF at Day 85 was analysed using MCP-Mod approach, including corresponding baseline as a covariate, with three pre-specified candidate models for the dose response (linear, Emax, and a Hill-Emax model). The testing of dose response signal was adjusted for multiplicity to control family-wise error rate at 0.10 level. Final model was selected among those indicated as significant based on the Akaike Information Criteria. Change from baseline in DAS28-CRP, RF, Vectra DA, CDAI, tender joint count, swollen joint count, patient's and physician's global assessment, and serum CRP were analysed using a mixed model for repeated measures (MMRM) analysis with corresponding baseline result included as a covariate.

### REFERENCES CITED IN THE DISCLOSURE

1. S. Sugio, A. Kashima, S Mochizuki, M. Noda, K. Kobayashi, Crystal structure of human serum albumin at 2.5 A resolution. Protein Eng 12, 439-446 (1999)
2. S. Lederman et al., Identification of a novel surface protein on activated CD4+ T cells that induces contact-dependent B cell differentiation (help). The Journal of experimental medicine 175, 1091-1101 (1992).
3. M. Croft, R. M. Siegel, Beyond TNF: TNF superfamily cytokines as targets for the treatment of rheumatic diseases. Nature reviews. Rheumatology 13, 217-233 (2017).
4. T. M. Foy, F. H. Durie, R. J. Noelle, The expansive role of CD40 and its ligand, gp39, in immunity. Seminars in immunology 6, 259-266 (1994).
5. J. B. Splawski, P. E. Lipsky, CD40-mediated regulation of human B-cell responses. Research in immunology 145, 226-234; discussion 244-229 (1994).
6. R. J. Armitage et al., Molecular and biological characterization of a murine ligand for CD40. Nature 357, 80-82 (1992).
7. P. Garside et al., Visualization of specific B and T lymphocyte interactions in the lymph node. Science 281, 96-99 (1998).
8. D. Hollenbaugh et al., The human T cell antigen gp39, a member of the TNF gene family, is a ligand for the CD40 receptor: expression of a soluble form of gp39 with B cell co-stimulatory activity. The EMBO journal 11, 4313-4321 (1992).
9. R. J. Noelle et al., A 39-kDa protein on activated helper T cells binds CD40 and transduces the signal for cognate activation of B cells. Proceedings of the National Academy of Sciences of the United States of America 89, 6550-6554 (1992).
10. T. M. Foy et al., gp39-CD40 interactions are essential for germinal center formation and the development of B cell memory. The Journal of experimental medicine 180, 157-163 (1994).
11. T. M. Foy et al., In vivo CD40-gp39 interactions are essential for thymus-dependent humoral immunity. II. Prolonged suppression of the humoral immune response by an antibody to the ligand for CD40, gp39. The Journal of experimental medicine 178, 1567-1575 (1993).
12. S. Han et al., Cellular interaction in germinal centers. Roles of CD40 ligand and B7-2 in established germinal centers. Journal of immunology 155, 556-567 (1995).
13. T. Kawabe et al., The immune responses in CD40-deficient mice: impaired immunoglobulin class switching and germinal center formation. Immunity 1, 167-178 (1994).
14. R. C. Allen et al., CD40 ligand gene defects responsible for X-linked hyper-IgM syndrome. Science 259, 990-993 (1993).
15. A. Aruffo et al., The CD40 ligand, gp39, is defective in activated T cells from patients with X-linked hyper-IgM syndrome. Cell 72, 291-300 (1993).
16. J. P. DiSanto, J. Y. Bonnefoy, J. F. Gauchat, A. Fischer, G. de Saint Basile, CD40 ligand mutations in x-linked immunodeficiency with hyper-IgM. Nature 361, 541-543 (1993).
17. D. T. Boumpas et al., A short course of BG9588 (anti-CD40 ligand antibody) improves serologic activity and decreases hematuria in patients with proliferative lupus glomerulonephritis. Arthritis and rheumatism 48, 719-727 (2003).
18. A. C. Grammer et al., Abnormal germinal center reactions in systemic lupus erythematosus demonstrated by blockade of CD154-CD40 interactions. The Journal of clinical investigation 112, 1506-1520 (2003).
19. W. Huang et al., The effect of anti-CD40 ligand antibody on B cells in human systemic lupus erythematosus. Arthritis and rheumatism 46, 1554-1562 (2002).
20. V. L. Patel, J. Schwartz, J. B. Bussel, The effect of anti-CD40 ligand in immune thrombocytopenic purpura. British journal of haematology 141, 545-548 (2008).
21. S. E. McKenzie et al., The role of the human Fc receptor Fc gamma RIIA in the immune clearance of platelets: a transgenic mouse model. Journal of immunology 162, 4311-4318 (1999).
22. J. E. Freedman, CD40-CD40L and platelet function: beyond hemostasis. Circulation research 92, 944-946 (2003).
23. L. Robles-Carrillo et al., Anti-CD40L immune complexes potently activate platelets in vitro and cause thrombosis in FCGR2A transgenic mice. Journal of immunology 185, 1577-1583 (2010).
24. J. S. Swers et al., Multivalent scaffold proteins as superagonists of TRAIL receptor 2-induced apoptosis. Molecular cancer therapeutics 12, 1235-1244 (2013).
25. R. Vazquez-Lombardi et al., Challenges and opportunities for non-antibody scaffold drugs. Drug discovery today 20, 1271-1283 (2015).
26. R. N. Gilbreth, B. M. Chacko, L. Grinberg, J. S. Swers, M. Baca, Stabilization of the third fibronectin type III domain of human tenascin-C through minimal mutation and rational design. Protein engineering, design & selection: PEDS 27, 411-418 (2014).
27. V. Oganesyan et al., Fibronectin type III domains engineered to bind CD40L: cloning, expression, purification, crystallization and preliminary X-ray diffraction analysis of two complexes. Acta crystallographica. Section F, Structural biology and crystallization communications 69, 1045-1048 (2013).
28. D. Muller et al., Improved pharmacokinetics of recombinant bispecific antibody molecules by fusion to human serum albumin. The Journal of biological chemistry 282, 12650-12660 (2007).
29. B. J. Smith et al., Prolonged in vivo residence times of antibody fragments associated with albumin. Bioconjugate chemistry 12, 750-756 (2001).
30. I. Berberich, G. L. Shu, E. A. Clark, Cross-linking CD40 on B cells rapidly activates nuclear factor-kappa B. Journal of immunology 153, 4357-4366 (1994).
31. M. Kaileh, R. Sen, NF-kappaB function in B lymphocytes. Immunological reviews 246, 254-271 (2012).
32. M. Roy et al., Studies on the interdependence of gp39 and B7 expression and function during antigen-specific immune responses. European journal of immunology 25, 596-603 (1995).
33. R. Ettinger et al., IL-21 induces differentiation of human naive and memory B cells into antibody-secreting plasma cells. Journal of immunology 175, 7867-7879 (2005).
34. J. L. Karnell et al., CD19 and CD32b differentially regulate human B cell responsiveness. Journal of immunology 192, 1480-1490 (2014).
35. B. R. Renshaw et al., Humoral immune responses in CD40 ligand-deficient mice. The Journal of experimental medicine 180, 1889-1900 (1994).
36. K. Kato et al., The soluble CD40 ligand sCD154 in systemic lupus erythematosus. The Journal of clinical investigation 104, 947-955 (1999).
37. R. K. Vakkalanka et al., Elevated levels and functional capacity of soluble CD40 ligand in systemic lupus erythematosus sera. Arthritis and rheumatism 42, 871-881 (1999).
38. P. Bird, J. E. Calvert, P. L. Amlot, Distinctive development of IgG4 subclass antibodies in the primary and secondary responses to keyhole limpet haemocyanin in man. Immunology 69, 355-360 (1990).
39. M. E. Devey, K. M. Bleasdale-Barr, P. Bird, P. L. Amlot, Antibodies of different human IgG subclasses show distinct patterns of affinity maturation after immunization with keyhole limpet haemocyanin. Immunology 70, 168-174 (1990).
40. J. Ferbas et al., A novel assay to measure B cell responses to keyhole limpet haemocyanin vaccination in healthy volunteers and subjects with systemic lupus erythematosus. British journal of clinical pharmacology 76, 188-202 (2013).
41. J. S. Miller et al., Diminished neo-antigen response to keyhole limpet hemocyanin (KLH) vaccines in patients after treatment with chemotherapy or hematopoietic cell transplantation. Clinical immunology 117, 144-151 (2005).
42. K. Streicher et al., The plasma cell signature in autoimmune disease. Arthritis & rheumatology 66, 173-184 (2014).
43. F. J. Dumont, IDEC-131. IDEC/Eisai. Current opinion in investigational drugs 3, 725-734 (2002).
44. W. Schuler et al., Efficacy and safety of ABI793, a novel human anti-human CD154 monoclonal antibody, in cynomolgus monkey renal allotransplantation. Transplantation 77, 717-726 (2004).
45. F. Langer et al., The role of CD40 in CD40L- and antibody-mediated platelet activation. Thrombosis and haemostasis 93, 1137-1146 (2005).
46. C. Heeschen et al., Soluble CD40 ligand in acute coronary syndromes. The New England journal of medicine 348, 1104-1111 (2003).
47. F. Mach, U. Schonbeck, G. K. Sukhova, E. Atkinson, P. Libby, Reduction of atherosclerosis in mice by inhibition of CD40 signalling. Nature 394, 200-203 (1998).
48. T. Oura et al., Long-term hepatic allograft acceptance based on CD40 blockade by ASKP1240 in nonhuman primates. American journal of transplantation: official journal of the American Society of Transplantation and the American Society of Transplant Surgeons 12, 1740-1754 (2012).
49. M. Watanabe et al., ASKP1240, a fully human anti-CD40 monoclonal antibody, prolongs pancreatic islet allograft survival in nonhuman primates. American journal of transplantation: official journal of the American Society of Transplantation and the American Society of Transplant Surgeons 13, 1976-1988 (2013).
50. D. Spencer et al., O-xylosylation in a recombinant protein is directed at a common motif on glycine-serine linkers. J Pharm Sci 102, 3920-3924 (2013).

We claim:

1. A method for suppressing a B cell-mediated immune response in a subject comprising:
administering a dose of between 500 mg to 3000 mg VIB4920 to a subject in need thereof; and
suppressing the B cell-mediated immune response.

2. The method of claim 1, wherein the dose is between 1000 mg and 1500 mg VIB4920.

3. The method of claim 2, wherein the dose is 1000 mg VIB4920.

4. The method of claim 2, wherein the dose is 1500 mg VIB4920.

5. The method of any of claims 1-4, wherein the dose is administered every 14 days or is administered twice per month.

6. The method of any of claims 1-5, wherein the dose is administered intravenously.

7. The method of any of claims 1-6, wherein the suppression of the B cell-mediated immune response is a reduction in antibody class switching.

8. The method of any of claims 1-6, wherein the suppression of the B cell-mediated immune response is a reduction in circulating B cells.

9. The method of any of claims 1-6, wherein the suppression of the B cell-mediated immune response is a reduction in plasma cell activity.

10. The method of any of claim 9, wherein the reduction in plasma cell activity is characterized by a reduction in plasma cell signature.

11. The method of claim 10, wherein the reduction in plasma cell signature is characterized by a reduction in expression of genes IGHA1, IGJ, IGKC, IGKV4-1, and TNFRSF17.

12. A method for treating an autoimmune disease or disorder comprising:
administering a dose of between 500 mg and 3000 mg VIB4920 to a patient in need thereof; and
treating the autoimmune disease or disorder.

13. The method of claim 12, wherein the dose is between 1000 mg and 1500 mg VIB4920.

14. The method of claim 13, wherein the dose is 1000 mg VIB4920.

15. The method of claim 14, wherein the dose is 1500 mg VIB4920.

16. The method of any of claims 12-15, wherein the dose is administered every 14 days or is administered twice per month.

17. The method of any of claims 12-16, wherein the dose is administered intraveneously.

18. The method of any of claims 12-17, wherein the treating the autoimmune disease or disorder is characterized by a reduction in markers of inflammation.

19. The method of claim 18, wherein the markers of inflammation comprise one or more of autoantibody levels, plasma cell (PC) signature, circulating B cells and antibody class switching.

20. The method of claim 18, wherein the treating is a reduction of clinical symptoms.

21. The method of claim 12, wherein the autoimmune disease or disorder is rheumatoid arthritis.

22. The method of claim 21, wherein the treating is a reduction of one or more of: rheumatoid factor (RF) autoantibodies, Vectra DA biomarker score, plasma cell (PC) signature, serum reactive C protein (CRP), DAS28-CRP, swollen joint counts, tender joint counts, or clinical disease activity index (CDAI).

23. The method of claim 22, wherein the treating is a reduction of rheumatoid factor autoantibodies.

24. The method of claim 23, wherein the reduction of RF autoantibodies is by at least 50% and is by no later than 85 days post-initiation of treatment.

25. The method of claim 22, wherein the treating is reduction of DAS28-CRP.

26. The method of claim 25, wherein the reduction of DAS28-CRP is an adjusted mean difference of at least −1.2.

27. The method of claim 25, wherein the reduction of DAS28-CRP is detectable following administration of a single dose of VIB4920.

28. The method of claim 22, wherein the treating is a reduction of Vectra DA biomarker score.

29. The method of claim 28, wherein the reduction of the Vectra DA biomarker score is an adjusted mean difference of at least −10.3.

30. The method of claim 12, wherein the autoimmune disease or disorder is one of systemic sclerosis, myositis, antiphospholipid syndrome, autoimmune hepatitis, lupus nephritis, idiopathic thrombocytopenia purpura, vasculitis, cutaneous lupus, autoimmune hemolytic anemia, Sjogren's disease, IgG4 related disease, or systemic lupus erythematosus.

31. The method of claim 30, wherein the treating is a reduction of clinical symptoms of the autoimmune disease or disorder.

32. The method of claim 30, wherein the treating is a reduction of PC signature.

33. The method of claim 32, wherein the reduction of PC signature is characterized by a reduction in expression of genes IGHA1, IGJ, IGKC, IGKV4-1, and TNFRSF17.

34. The method of claim 30, wherein the treating is a reduction in one or more biomarkers of the autoimmune disease or disorder.

35. A method for reducing a measure of rheumatoid arthritis (RA) disease activity in a patient being treated for rheumatoid arthritis comprising:
administering VIB4920 to the patient;
wherein the measure of RA disease activity comprises one or more of DAS28-CRP, clinical disease activity index (CDAI), patient's global assessment or physician's global assessment;
wherein the VIB4920 is administered at a dose of between approximately 500 mg and 3000 mg; and
reducing the measure of RA disease activity in the patient.

36. The method of claim 35, wherein the VIB4920 is administered at a dose of between approximately 1000 mg and 2000 mg.

37. The method of claim 36, wherein the VIB4920 is administered at a dose of between approximately 1000 mg and 1500 mg.

38. The method of claim 37, wherein the VIB4920 is administered at a dose of approximately 1000 mg.

39. The method of claim 37, wherein the VIB4920 is administered at a dose of approximately 1500 mg.

40. The method of any of claims 35-39, wherein the VIB4920 is administered every 14 days or is administered twice per month.

41. The method of any of claims 35-40, wherein the VIB4920 is administered intravenously.

42. The method of any of claims 35-41, wherein the measure is DAS28-CRP and the reducing is at least an adjusted mean change of −1.2.

43. The method of claim 42, wherein the reducing is at least an adjusted mean change of −2.2.

44. The method of any of claims 35-41, wherein the measure is DAS28-CRP and the reducing is observed following a first dose of VIB4920.

45. A method for reducing rheumatoid factor (RF) autoantibodies in a patient in treatment for rheumatoid arthritis comprising:
administering VIB4920 at a dose of between approximately 500 mg and 3000 mg to the patient; and
reducing RF autoantibodies in the patient.

46. The method of claim 45, wherein the dose is between approximately 1000 mg and approximately 2000 mg.

47. The method of claim 46, wherein the dose is between approximately 1000 mg and approximately 1500 mg.

48. The method of claim 47, wherein the dose is approximately 1000 mg.

49. The method of claim 48, wherein the dose is approximately 1500 mg.

50. The method of any of claims 45-49, wherein the dose is administered every 14 days or is administered twice per month.

51. The method of any of claims 45-50, wherein the dose is administered intravenously.

52. The method of any of claims 45-51, wherein the RF autoantibodies are reduced by at least 40%.

53. The method of any of claims 46-51, wherein the RF autoantibodies are reduced by at least 50%.

54. The method of any of claims 46-53, wherein the RF autoantibodies are reduced by no later than 85 days post initiation of treatment.

55. A method for reducing a biomarker score in a patient in treatment for rheumatoid arthritis comprising:
administering approximately 500 mg to 3000 mg VIB4920 to the patient,
wherein the biomarker score is one or more of plasma cell (PC) gene signature, Vectra-DA score, or serum C reactive protein level (CRP); and
reducing the biomarker score in the patient.

56. The method of claim 55, wherein the dose is between approximately 1000 mg and approximately 2000 mg.

57. The method of claim 56, wherein the dose is between approximately 1000 mg and approximately 1500 mg.

58. The method of claim 57, wherein the dose is approximately 1000 mg.

59. The method of claim 58, wherein the dose is approximately 1500 mg.

60. The method of any of claims 55-59, wherein the dose is administered every 14 days or is administered twice per month.

61. The method of any of claims 55-60, wherein the dose is administered intravenously.

62. The method of any of claims 55-61, wherein the biomarker score is Vectra DA and the reducing is an adjusted mean difference of at least −10.3.

63. The method of any of claims 57-61, wherein the biomarker score is serum CRP.

64. A method for reducing plasma cell (PC) gene signature scores in a patient in need thereof, comprising:
administering VIB4920 to a patient in need thereof,
wherein the patient is being treated for systemic lupus erythematosus, rheumatoid arthritis, myositis, antiphospholipid syndrome, autoimmune hepatitis or Sjogren's disease, and
wherein the VIB4920 is administered at a dose of approximately 500 mg to 3000 mg; and
reducing the PC gene signature score in the patient.

65. The method of claim 64, wherein the dose is between approximately 1000 mg and approximately 2000 mg.

66. The method of claim 65, wherein the dose is between approximately 1000 mg and approximately 1500 mg.

67. The method of claim 66, wherein the dose is approximately 1000 mg.

68. The method of claim 67, wherein the dose is approximately 1500 mg.

69. The method of any of claims 64-68, wherein the dose is administered every 14 days or is administered twice per month.

70. The method of any of claims 64-69, wherein the dose is administered intravenously.

71. A method of reducing autoantibodies in a patient in treatment for an autoimmune disorder comprising:
administering VIB4920 to a patient in need thereof,
wherein the patient is being treated for an autoimmune disease characterized by presence of autoantibodies; and
wherein the VIB4920 is administered at a dose of approximately 500 mg to 3000 mg; and
reducing the autoantibodies in the patient.

72. The method of claim 71, wherein the dose is between approximately 1000 mg and approximately 2000 mg.

73. The method of claim 72, wherein the dose is between approximately 1000 mg and approximately 1500 mg.

74. The method of claim 73, wherein the dose is approximately 1000 mg.

75. The method of claim 73, wherein the dose is approximately 1500 mg.

76. The method of any of claims 71-75, wherein the dose is administered every 14 days or is administered twice per month.

77. The method of any of claims 71-76, wherein the dose is administered intravenously.

78. The method of any of claims 71-77, wherein the autoimmune disease is systemic lupus erythematosus, rheumatoid arthritis, myositis, antiphospholipid syndrome, autoimmune hepatitis or Sjogren's disease.

79. A method of reducing inflammation in a patient comprising:
administering VIB4920 to a patient in need thereof,
wherein the patient is being treated for an inflammatory disease or disorder, or is being prophylactically treated for anticipated inflammation in response to an organ or tissue transplant; and
wherein the VIB4920 is administered at a dose of approximately 1000 mg to 3000 mg; and
reducing inflammation in the patient.

80. The method of claim 79, wherein the dose is between approximately 1000 mg and approximately 2000 mg.

81. The method of claim 80, wherein the dose is between approximately 1000 mg and approximately 1500 mg.

82. The method of claim 79, wherein the dose is approximately 1000 mg.

83. The method of claim 79, wherein the dose is approximately 1500 mg.

84. The method of claim 79, wherein the dose is approximately 3000 mg.

85. The method of any of claims 79-84, wherein the dose is administered every 14 days or is administered twice per month.

86. The method of any of claims 79-85, wherein the dose is administered intravenously.

87. The method of claim 84, wherein the dose is administered once per month.

88. The method of any of claims 79-87, wherein the patient is being treated in conjunction with or is being prophylactically treated to prevent rejection of an organ or tissue transplant.

89. The method of any of claims 79-87, wherein the inflammatory disease or disorder is an inflammatory myopathy, or is lupus nephritis, cutaneous lupus, RA, SLE, ITP, myositis, Sjogren's syndrome, vasculitis, systemic sclerosis, autoimmune hemolytic anemia, myasthenia gravis or focal segmental glomerulosclerosis.

90. The method of claim 21, wherein the treating is achieving ACR20, ACR50, or ACR70.

91. The method of any of claims 1-11, wherein the suppressing the B cell-mediated immune response is long-lasting.

92. The method of any of claims 12-34 or 90, wherein the treating the autoimmune disease or disorder is long-lasting.

93. The method of any of claims 35-44, wherein the reducing the measure of RA disease activity in the patient is long-lasting.

94. The method of any of claims 45-54, wherein the reducing RF autoantibodies in the patient is long-lasting.

95. The method of any of claims 55-63, wherein the reducing the biomarker score in the patient is long-lasting.

96. The method of any of claims 64-70, wherein the reducing the PC gene signature score in the patient is long-lasting.

97. The method of any of claims 71-78, wherein the reducing the autoantibodies in the patient is long-lasting.

98. The method of any of claims 79-89, wherein the reducing inflammation in the patient is long-lasting.

99. A method of inducing immune tolerance to a replacement therapy in a patient comprising:
administering VIB4920 to a patient in need of a replacement therapy,
wherein the VIB4920 is administered at a dose of approximately 1000 mg to 3000 mg; and
inducing immune tolerance to the replacement therapy in the patient.

100. The method of claim 99, wherein the VIB4920 is administered at a dose of approximately 1500 mg to 3000 mg.

101. The method of claim 100, wherein the VIB4920 is administered at a dose of approximately 2500 mg to 3000 mg.

102. The method of claim 101, wherein the VIB4920 is administered at a dose of approximately 3000 mg.

103. The method of any one of claims 99-102, wherein the dose is administered approximately once every two to four weeks.

104. The method of claim 103, wherein the dose is administered approximately once every four weeks.

105. The method of claim 102, wherein the dose in administered once per month.

106. The method of any of claim 99, 102, or 105, wherein the replacement therapy is a protein or peptide.

107. The method of claim 106, wherein the inducing immune tolerance comprises a reduction in production of neutralizing antibodies to the protein or peptide by the patient.

108. The method of claim 107, wherein the protein is Factor VIII and the patient is a hemophilia patient.

109. The method of claim 107, wherein the protein is Factor IX and the patient is a hemophilia patient.

110. The method of claim 108, wherein the inducing immune tolerance is a reduction in neutralizing anti-Factor VIII antibodies in the patient.

111. The method of claim 106, wherein the protein or peptide is an enzyme.

112. The method of claim 111, wherein the enzyme is agalsidase alfa or agalsidase beta and the patient is a Fabry disease patient.

113. The method of claim 111 wherein the enzyme is idursulfase and the patient is a mucopolysaccharidosis II or Hunter syndrome patient.

114. The method of claim 111, wherein the enzyme is iaronidase and the patient is a mucopolysaccharidosis I syndrome patient.

115. The method of claim 111, wherein the enzyme is alglucosidase alpha and the patient is a Pompe disease patient.

116. The method of any of claim 99, 102, or 105, wherein the replacement therapy is a viral vector comprising a nucleic acid encoding a therapeutic peptide or protein.

117. The method of claim 116, wherein the inducing immune tolerance to the replacement therapy comprises a reduction in immune response to the viral vector, a reduction in neutralizing antibodies to the therapeutic protein, or both in the patient.

118. The method of claim 117 wherein the viral vector is adeno-associated virus (AAV).

119. The method of claim 118, wherein the inducing immune tolerance comprises a reduction in immune response to the AAV.

120. The method of claim 119, wherein the reduction in immune response to the AAV is a reduction in a T cell response to the AAV or a reduction in antibodies to the AAV.

121. The method of claim 120, wherein the reduction in T cell response is a reduction in T cell response to AAV capsid protein.

122. The method of claim 117, wherein the inducing immune tolerance to the replacement therapy comprises the reduction in neutralizing antibodies to the therapeutic protein.


SEQUENCE LISTING (6 sequences)

SEQ ID NO: 1 - protein, 785 aa, Artificial Sequence
  synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 83 aa, Artificial Sequence
  synthetic construct
IEVKDVTDTTALITWSDDFGEYVWCELTYGIKDVPGDRTTIDLWYHHAHYSIGNLKPDTEYEVSLICRRGDMSSNPAKETFTT

SEQ ID NO: 3 - protein, 83 aa, Artificial Sequence
  synthetic construct
IEVKDVTDTTALITWSDDFGEYVWCELTYGIKDVPGDRTTIDLWYHHAHYSIGNLKPDTEYEVSLICRSGDMSSNPAKETFTT

SEQ ID NO: 4 - protein, 190 aa, Artificial Sequence
  synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGSGGGGSGGGGSRLDAPSQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 5 - protein, 83 aa, Artificial Sequence
  synthetic construct
IEVKDVTDTTALITWSDEFGHYDGCELTYGIKDVPGDRTTIDLWWHSAWYSIGNLKPDTEYEVSLICRRGDMSSNPAKETFTT

SEQ ID NO: 6 - protein, 190 aa, Artificial Sequence
  synthetic construct
SQIEVKDVTDTTALITWSDEFGHYDGCELTYGIKDVPGDRTTIDLWWHSAWYSIGNLKPDTEYEVSLICYTDQEAGNPAKETFTTGGGGSGGGGSGGGGSRLDAPSQIEVKDVTDTTALITWSDEFGHYDGCELTYGIKDVPGDRTTIDLWWHSAWYSIGNLKPDTEYEVSLICYTDQEAGNPAKETFTT